(19) United States
(12) Patent Application Publication
(10) Pub. No.: US 2025/0256100 A1
(43) Pub. Date: Aug. 14, 2025

(54) METHODS FOR PRESERVING VISUAL FUNCTION USING RAMP WAVEFORM ELECTRICAL STIMULATION

(71) Applicants: The Schepens Eye Research Institute, Inc., Boston, MA (US); Oslo University Hospital, Oslo (NO)

(72) Inventors: Dong Feng Chen, Newtonville, MA (US); Menglu Yang, Littleton, MA (US); Anton Lennikov, Stoneham, MA (US); Tor Paaske Utheim, Oslo (NO); Karen Chang, Malden, MA (US); Ajay Ashok, Melrose, MA (US); Darlene Dartt, Newton, MA (US); Kin-Sang Cho, Winchester, MA (US)

(21) Appl. No.: 18/857,590

(22) PCT Filed: Apr. 18, 2023
(86) PCT No.: PCT/US2023/018921

§ 371 Date: Oct. 17, 2024

Related U.S. Application Data
(60) Provisional application No. 63/429,445, filed on Dec. 1, 2022.
(60) Provisional application No. 63/336,234, filed on Apr. 28, 2022.
(60) Provisional application No. 63/332,520, filed on Apr. 19, 2022.

(51) Int. Cl. A61N 1/36; A61N 1/05

(52) U.S. Cl. CPC A61N 1/36046; A61N 1/0543

(57) ABSTRACT

Provided herein are methods for treating macular degeneration, age-related macular degeneration, and photoreceptor degeneration in a subject including delivering to the subject electrical stimulation in the form of a ramp waveform, wherein the electrical stimulation is delivered through the skin around the eye. The methods provide for electrical stimulation at least most days per week.

### CLAIM OF PRIORITY

This application claims the benefit of U.S. Provisional Patent Application Ser. No. 63/332,520, filed on Apr. 19, 2022, U.S. Provisional Patent Application Ser. No. 63/336,234, filed on Apr. 28, 2022, and U.S. Provisional Patent Application Ser. No. 63/429,445, filed on Dec. 1, 2022. The entire contents of the foregoing are hereby incorporated by reference.

### TECHNICAL FIELD

The present disclosure relates to treatments for ocular disorders.

### BACKGROUND

Transcorneal electrical stimulation (TcES) is increasingly applied as a therapy for preserving and improving vision in retinal neurodegenerative and ischemic disorders. However, a common complaint about TcES is its induction of clinical dry eye symptoms. Thus there remains a need to further develop treatments for retinal neurodegenerative conditions that do not cause dry eye.

### SUMMARY

Provided herein are methods of treating macular degeneration, age-related macular degeneration, photoreceptor degeneration, ischemic retinopathy, diabetic retinopathy, ischemic optic neuropathy, traumatic optic neuropathy, and/or glaucoma in a subject comprising delivering electrical stimulation in a ramp waveform to the eye of the subject.

Provided herein are methods of treating corneal abrasions, corneal ulcers, dry eye syndrome, corneal neuropathic pain, neuropathic keratitis, and corneal vascularization in a subject comprising delivering electrical stimulation in a ramp waveform to an eye of the subject.

Provided herein are methods of preserving visual function in an injured or diseased eye of a subject comprising delivering electrical stimulation in a ramp waveform to the eye of the subject.

Provided herein are methods of increasing nerve length in a cornea of an injured or diseased eye of a subject comprising delivering electrical stimulation in a ramp waveform to the eye of the subject.

Provided herein are methods of repressing microglia activation in an injured or diseased eye of a subject comprising delivering electrical stimulation in a ramp waveform to the eye of the subject.

Provided herein are methods of preserving tear production in an injured or diseased eye of a subject comprising delivering electrical stimulation in a ramp waveform to the eye of the subject.

In some embodiments, the electrical stimulation is delivered transpalpebrally. In some embodiments, the ramp waveform is approximately 100 ms in duration. In some embodiments, the ramp waveform is delivered at approximately 20 Hz. In some embodiments, the electrical stimulation comprises both a ramp waveform and a rectangular waveform. In some embodiments, the electrical stimulation is delivered every day or every other day. In some embodiments, the electrical stimulation is delivered at least three days at least four days, at least five days, or at least six days per week. In some embodiments, the electrical stimulation further comprises days in which no electrical stimulation is delivered. In some embodiments, the electrical stimulation is delivered for at least one minute, two minutes, three minutes, four minutes, five minutes, six minutes, seven minutes, eight minutes, nine minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes, or 30 minutes per day. In some embodiments, the electrical stimulation is delivered for no more than 30 minutes, 29 minutes, 28 minutes, 27 minutes, 26 minutes, 25 minutes, 24 minutes, 23 minutes, 22 minutes, 21 minutes, 20 minutes, 19 minutes, 18 minutes, 17 minutes 16 minutes, 15 minutes, 14 minutes, 13 minutes, 12 minutes, 11 minutes, 10 minutes, nine minutes, eight minutes, seven minutes, six minutes, five minutes, four minutes, three minutes, two minutes, or one minute per day. In some embodiments, the electrical stimulation comprises amplitude priority stimulation. In some embodiments, the electrical stimulation comprises about 100 μA to about 500 μA. In some embodiments, the electrical stimulation comprises about 300 μA. In some embodiments, the electrical stimulation comprises about 100 μA. In some embodiments, the preserving visual function in a subject comprises preserving visual acuity and/or contrast sensitivity. In some embodiments, the preserving visual function in a subject comprises preserving positive scotopic threshold response in the subject. In some embodiments, the preserving visual function in a subject comprises preserving an ERG a wave in a subject. In some embodiments, the repressing microglia activation comprises repressing angiogenic processes and/or inducing anti-angiogenic processes.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-F Recording of ES waveforms in the posterior segment of the eye in vivo. (A) Graphical summary of the electrical stimulation and waveform recording (left panel) and conductive resistance recording in the post-mortem eyes (right panel). (B-D) Pulse generator settings (top) and oscilloscope recordings in the subretinal space (bottom) of (B) rectangular, (C) Sine and (D) ramp tES waveforms; (E) Quantitative analysis of the electric field potential recordings in the subretinal space; n=4/group. Statistical significance was determined using one-way ANOVA with Tukey multiple comparisons. ***p<0.001; value=means±S.D.; (F) Resistance recording from cornea to subretinal space in post-mortem human and mouse eyes (human eyes n=3; mouse eyes n=6). Statistical significance was determined using one-way ANOVA with Tukey multiple comparisons. ***p<0.001; value=means±S.D.

FIGS. 2A-E Therapeutic transpalbabral ES attenuates laser-induced choroidial neovascularization in mice. Fundus images (A), fluorescein angiography (B) and (C) retinal OCT imaging of mice (6-8 weeks old) that received laser eye injury (523 nm laser; 150 mW; 50 μm; 100 ms) followed by transpalbabral sham- or ES-treatment (300 μA, 20 Hz) 4 minutes/day for 7 days. (D) Quantification of fluorescence size in FA images. n=8/group. (E) Surface area of the central cross section of the laser CNV spots by OCT. n=4/group (4 spots per animal were averaged) *p<0.05 by Student's t-test; value=means±S.D.

FIGS. 3A-H Transpalpebral ES limits inflammatory and angiogenesis responses after the laser eye injury. (A) Epifluorescent photomicrographs of RPE/Choroidal/Scleral whole-mounts from Sham-control and ES-treated laser-injured mice that were immunolabeled for markers of retinal vessel endothelial cells (Lectin). (B) Quantification of isolectin-stained areas as indicators of neovascularization. Statistical significance was determined by Student's t-test. *p<0.001; value=means±S.D. n=3. (C) Epifluorescent photomicrographs inmmunolabeled for microglia/macrophage (IBA-1). (D) Quantification of IBA1+ cells. Statistical significance was determined by Student's t-test. *p<0.001; value=means±S.D. n=3. (E) Epifluorescent photomicrographs of scleral whole-mounts from control and ES-treated laser-injured mice that were immunolabeled for markers of activated microglia/macrophage (COX-2). (F) Quantification of COX2+ cells/field. Statistical significance was determined by Student's t-test. *p<0.001; value=means±S.D. n=3. (G) Epifluorescent photomicrographs immunolabeled for angiogenesis (VEGFa). (H) Quantification of VEGFa+ cells per area micrometers². Statistical significance was determined by Student's t-test. *p<0.001; value=means±S.D. n=3. Nuclei counterstained with DAPI (blue). Scale bar 50 μm.

FIGS. 4A-E ES reduce microglia activation through modulation of Ca²⁺ currents. qPCR analysis of COX-2, IL-1β, and IL6 after ES followed by ATP 10⁻⁷ M stimulation (n=3). The Fura-2 AM Ca²⁺ curve and [Ca²⁺]ᵢ quantification in ES treated cells in response to ATP 10⁻⁷ M stimulation with Ca²⁺ present in the KRB buffer (B, C) (n=10/group) and Ca²⁺-free KRB buffer (n=8/group) (D, E). Statistical significance was determined one-way ANOVA with Tukey multiple comparisons. *p<0.05;**p<0.01; value=means±S.D.

FIGS. 5A-E ES induces mitochondria depolarization, reduced ATP production, and metabolism in primary microglia. JC-1 mitochondrial polarization staining (A) and JC-1 dimers to monomers ratios (B) in control, ES-treated and FCCP-treated microglia cells. Statistical significance was determined using one-way ANOVA with Tukey multiple comparisons. ***p<0.001; ****p<0.0001; value=means±S.D. (C, D) Results of Agilent Seahorse oxygen consumption rate and OCR rates for Basal respiration, maximum respiration, proton leak, and ATP production. Statistical significance was determined by Student's t-test. ns p>0.05; *p<0.05;**p<0.01; value=means±S.D (E) Microglia cells bulk ATP content. Statistical significance was determined by Student's t-test. *p<0.05;**p<0.01; value=means±S.D.

FIGS. 6A-C Microglia cell's cell membrane potential in control cells (A) and depolarization visualized by FluoVolt after 30 min of ES (B). The images represent 488 nm excitation fluorescence with applied heatmap. (C) A graph of the mean intensity for electrical stimulation and control conditions over time. Statistical significance was determined by two way Anova. *p<0.05; value=means±S.D. n=3 independent experiments per time point; 20+ cells quantified per experiment.

FIGS. 7A-F Electric stimulation reduces migration and tube formation of primary human retinal endothelial cells. The scratch assay (A) in control and ES cultures. (B) Quantification of the scratch surface area after 24 hours post scratch. n=10/group for 0 h, 20/group for 24 h. (C) Tube formation assay in control and ES cultures. Quantitative analysis of nodes (D), junctions (E), and total tubule length (F). n=10/group. Statistical significance was determined by Student's t-test. ***p<0.001; value=means±S.D.

FIGS. 8A-B Electric stimulation reduce RPE/Choroidal explants endothelial cells outgrowth. (A) The control and ES explants after 7-days of culture. (B) Quantitative analysis of the endothelial cells outgrowth from the explant. Statistical significance was determined by Student's t-test. *p<0.05; value=means±S.D. n=5/group.

FIGS. 9A-C Electric stimulation reduce VEGF expression the endothelial cells. (A) Immunolabeling of the VEGF in the control and ES-treated HREC 12 hours post-stimulation. (B) Western blotting and (C) densitometry analysis of HREC control and ES cultures. Statistical significance was determined by Student's t-test. ***p<0.001; value=means±S.D. n=3/group.

FIGS. 10A-D TcES induced corneal epithelial damage after 14 days of continuous application in mice. Sodium fluorescein dye was applied to each eye 14 days after TcES, and images were taken under slit lamp microscopy before (A, left panel) and after (A, right panel) sodium fluorescein dye application. Representative images were shown in A. In eyes without TcES, minimum to no fluorescein stain was presented (A, top panel); in the eyes treated with TcES, medium to severe fluorescein stain was presented (A, bottom panel). The fluorescein stain was scored (B), and the mean value of the scores was analyzed (C). The tear production from each eye was measured and plotted in (D). Each data point is the mean value of one cornea. **p<0.01; n.s. p>0.05 (n=4 eyes/group).

FIGS. 11A-C Decreased immunoreactivity of MUC4 was detected in the corneas subjected to TcES. The mice corneas were harvested after 14 days of ES and IF on the cornea. The whole mount was conducted in non-treated eyes (A, left panel) and TcES-treated eyes (A, right panel). The immunoreactivity of MUC4 was detectable throughout the cytoplasm in corneal epithelial cells located in the top layer of the stratified squamous epithelium (A). WB analysis was conducted using corneas undergone TcES (n=4) or those of the contralateral control eyes (n=4) (B). A single band at 150 kDa was detected in all samples (B). The ECL signal from each band was normalized to ß-tubulin, and the quantification result was plotted in (C). *p<0.05.

FIGS. 12A-D ES inhibited MUC4 and ZO-1 expression in primary corneal epithelial cells. Primary corneal epithelial cells were cultured on coverslips till confluency. ES was applied 30 min each day for 3 days, and immunofluorescence microscopy was performed with cultured goblet cells using antibodies against MUC4 (A) and ZO-1 (B). The panels on the left are representative images from control, while the panels on the right are representative images from cells treated with ES. Protein samples were collected from each group's cell pellets, and Western Blot analysis was performed using antibodies against MUC4 (A) and ZO-1 (B). For MUC4, 3 lanes on the left indicate proteins extracted from control, and 3 lanes on the right indicate proteins extracted from the ES-treated cells. For ZO-1, 4 lines on the left indicate proteins extracted from control, and 4 lines on the right indicate proteins extracted from the ES-treated cells. The quantified relative abundance of MUC4 (C) and ZO-1 (D) is plotted and analyzed. *p<0.05. **p<0.01.

FIGS. 13A-C TcES negatively regulates the secretory mucin release by inhibiting intracellular [Ca²⁺]ᵢ signaling in conjunctival goblet cells. After 14 days of TcES, the whole conjunctiva (n=3 for each group) of the mice are dissected and incubated in the tear buffer for 4 hours in the presence of Cch (10⁻⁴ M). After 4 h incubation, the supernatant of each conjunctiva was collected, and the level of MUC5AC was detected using ELISA (A). The amount of MUC5AC was plotted in (B). Primary human conjunctival goblet cells were treated with ES for 1 hour, and then the [Ca²⁺]ᵢ mobilization was induced by Cch (10⁻⁴ M) and measured in cells treated with or without ES (C). Each data point indicates a single individual assay. *p<0.05. **p<0.01.

FIGS. 14A-D TpES did not induce corneal epithelial damage while increased tear production after 14 days of continuous application in mice. Sodium fluorescein dye was applied to each eye after TpES 4 min per day for 14 days, and images were taken under slit lamp microscopy before (A, left panel) and after (A, right panel) sodium fluorescein dye application. Representative images were shown in A. Minimum to no fluorescein stain was present in both control (top) and TpES eyes (bottom). The fluorescein stain was scored (B), and the mean value of the scores was analyzed (C). The tear production from each eye plotted in (D). Each data point is the mean value of one cornea. *p<0.05; n.s. p>0.05.

FIGS. 15A-B TpES did not induce alter the MUC4 expression in cornea. WB analysis was conducted using corneas undergone TpES (n=4) or those of the contralateral control eyes (n=4) (A). A single band at 150 kDa was detected in all samples (A). The 655 ECL signal from each band was normalized to ß-tubulin, and the quantification result was plotted in (B). n.s. p>0.05.

FIGS. 16A-B TcES presented higher conductance resistance to the retina than TpES. The resistance from the surface of the cornea to the retina (Rc, A left panel) and the total resistance from the orbital skin to the retina (Rs, A right panel) of the same eye were measured, and each measurement was plotted in (B). ****p<0.0001.

FIGS. 17A-D Electric Stimulation promotes photoreceptor survival in human retinal explant cultures. (A-C) Representative images of retinal explants in (A) Control, (B) rectangular ES or (C) ramp ES treated cultures that were immunolabeled for rhodopsin and counterstained with a nuclear marker DAPI. (D) Quantitative analysis of rhodopsin+ cells in human retinal explants. Retinal explants were obtained from the three individuals, and the results of three explants per individual were averaged. Statistical significance was determined using an unpaired t-test; *p<0.05.

FIGS. 18A-E Combined rectangular and ramp tES improves ERG and OMR responses in Rho−/− mice. (A) The schematics of the experimental timeline. (B) The control ERG b-wave in photopic M-cone electroretinography (ERG) in 8 weeks old Rho−/− mice (left) and after two weeks of tES using a combination of rectangular and ramp waveforms (night). (C, D) Quantification of ERG b wave amplitude of photopic M-cone (C) and S-cone (D) responses of 8 weeks old Rho−/− mice with and without tES; (E) Visual acuity (VA) of Rho−/− mice at 6 and 8 weeks of age. Mice treated with tES showed significant improvement in VA compared to the sham-treated mice. ERG and OMR quantifications (tES group n=12; Sham group n=9). Statistical significance was determined using an unpaired t-test; *p<0.05.

FIGS. 19A-F TES increases the survival of photoreceptors. (A, B) Representative images of retinal sections immunolabeled for (A) Blue-opsins and (B) Red/Green-opsins, respectively, with DAPI counterstaining. Scale bar=50 μm. (C-E) Quantification of (C) B-opsins positive cells; (D) R/G-opsins positive cells; (E) B-opsins and R/G-opsins positive cells numbers combined in the retina sections of tES or sham-treated eyes. The data averaged from the quantification of 10 sections per mouse (tES group n=3; Sham group n=3). Statistical significance was determined using an unpaired t-test; *p<0.05; **p<0.01 (F). The quantification of the retinal ONL thickness in tES or sham-treated eyes. The data averaged from the quantification of 10 sections per mouse (tES group n=6; Sham group n=5). Statistical significance was determined using an unpaired t-test; ***p<0.01.

FIGS. 20A-C TES increases bipolar cell sprouting. (A, B) Representative images of retinal sections immunolabeled for PKCα and DAPI in sham control (A) and TES-treated (B) groups. Scale bar=20 μm. GCL—ganglion cell layer; IPL—inner plexiform layer; INL—inner nuclear layer; OPL—outer plexiform layer; ONL—outer nuclear layer; PS—photoreceptor layer. The dashed line indicates the inner edge of the ONL. The white box demonstrates the enlarged PKCα positive bipolar cell sprouting into the ONL. The white dotted line indicates the margin between OPL and ONL in enlarged panels. (C) Quantification of bipolar cell processes extending into the ONL. Retina sections from TES-treated eyes exhibited significantly increased bipolar cell processes compared to the sham-treated group. ***p<0.001, unpaired t-test. (tES group n=7; Sham group n=5). **p<0.01; ***p<0.001, unpaired t-test

FIGS. 21A-I Microcurrent ES of ramp waveform inhibits BV-2 cell migration. (A-C) Schematic representation of different ES waveforms: (A) Ramp; (B) Rectangular; (C) Sine. (D-G) Photomicrographs of migratory BV-2 cells stained by a fluorescence vital dye Calcein AM (green) in the scratch assay and assessed at 0 and 48 h post scratching. Cells were cultured under a control condition (D) or subjected to ES of ramp (E), rectangular (F), or sine (G) waveform. Scale bar: 100 m. (H,I) Quantitative analysis of scratch distance (H) and scratch surface area (I). n=8 cultures/group; Statistical significance was determined using one-way ANOVA with Tukey multiple comparisons. *p<0.05; **p<0.01; value=means±S.D.

FIGS. 22A-F Ramp ES inhibits the redistribution of cytoskeletal and motility proteins and the formation of cellular lamellipodia and microvilli. (A-D) Immunostaining of F-actin (red) and Q-tubulin (green) in BV-2 cells cultured under a control condition (A) after ES biphasic ramp; white arrow indicates redistribution of F-actin within the cell. (B), ES biphasic rectangular (C), or ES biphasic sine (D). Scale bar: 20 μm. (E, F) Scanning electron microscopy images of BV-2 cells in control (E) and after ES biphasic ramp (F). Inserts reveal cell membrane morphology in the leading edge and lamellipodia. Scale bar: 5 m; insert: 1 μm.

FIGS. 23A-C Ramp ES reduces phagocytosis of BV-2 cells. Representative fluorescent images of BV-2 cells cultured under a control condition or ES biphasic ramp (A) and incubated with fluorescent (cy3) zymosan particles (red) for 24 h and Calcein AM stain (green) was used to visualize BV-2 cells. Scale bar: 50 μm. Inserts present individual cells with zymosan particles. Quantification of zymosan particles in BV-2 cells in control and ramp ES-treated cultures per field (B) and the average number of zymosan particles per cell (C); Control n=17; Ramp n=14. Statistical significance was determined by Student's t-test. **p<0.01; value=means±S.D.

FIGS. 24A-D Electric stimulation with ramp waveform reduces BV-2 cell activation in response to LPS stimulation. (A) Heatmap of inflammatory cytokine levels as measured by values of densitometry of cytokine arrays and presented as fold changes normalized to the value of the LPS-stimulated group without the ES. RT-PCR (B-D) demonstrated changes in levels of expression of TNF α (B), IL-6 (C), and COX-2 (D) in BV-2 cells subjected to ES biphasic ramp at 24 h after LPS stimulation. n=3; Statistical significance was determined using one-way ANOVA with Tukey multiple comparisons. *p<0.05; **p<0.01; value=means±S.D.

FIGS. 25A-F RNA-seq analysis for the transcriptome profiles of LPS-challenged BV-2 cells with or without ES-treatment. (A) Heatmap of DEGs in Control, LPS, and Ramp ES+LPS treated groups; the values were normalized as fold change over the control group. (B, C) Volcano plots of Control vs LPS (B) and LPS vs Ramp ES+LPS (C). (D-F) Dot plots of top ten GO terms through analysis of ES-suppressed genes that were divided into the categories of Biological process (D), Cellular components (E), and Molecular function (F).

FIGS. 26A-B Developmental downregulation of TETs in RGCs. (A) Quantification of TET1, TET2 and TET3 mRNA with qPCR in the developing mouse RGCs. Note the significantly decreased mRNA levels of all TET(s) with age. Retinas from 4 mice were pooled into two groups (represented as two data points) for the E-14 age group. Values are means±SEM. *p≤0.05; **p≤0.01; ***p≤0.001 compared to E-14; #p<0.05; ##p<0.01 compared to P-0 by Student t test. (B) Images of retinal sections of E16, P0 and adult mice (3 months) double-immunolabeled with primary antibodies against 5mc (red) and 5hmc (green); retinal sections were counterstained with a nuclear marker DAPI (blue). INL: Inner Nuclear Layer; GCL: Ganglion Cell Layer.

FIGS. 27A-E Nerve growth factor induces PC12 differentiation and TET1 expression. (A) Bright field images of PC-12 cells exposed to increasing concentrations of NGF (0, 5 and 100 ng/mL) exhibited a dose dependent increase in differentiation as indicated by significantly increased neurite outgrowth quantified by (B) neurite length and (C) neurite numbers per field. Images were acquired 5 days post NGF induction of differention. (D) Results of qPCR assessments of the levels of TET1, TET2 and TET3 in PC-12 cells exposed to increasing concentrations of NGF. A significant dose dependent increase in TET1 nRNA was observed in PC12 cells. RNA samples were collected 24 hours post NGF treatment for qPCR analysis. (E) Cells transfected with TET1-CD showing increased neurite outgrowth compared to vehicle treated cells. Images were acquired 5 days post transfection. Values are means±SEM. *p≤0.05; ***p≤0.001 by Student t test.

FIGS. 28A-F Increased levels of TET1 correlates with neurite outgrowth in PC12 cells (A-C) ES increased neurite outgrowth in PC12 cells compared to control unstimulated cells. PC12 cells incubated with a low level of NGF (5 ng/mL) (panel 1 and 2) and received 20 min ES at 24 hr post NGF induction. (D,E) Result of qPCR assessing mRNA levels of GAP43 (D) and TET1 (E) expression in Non-ES- and ES-treated cells. (F) Immunocytochemistry based analysis of 5hmc (green) expression, an intermediate product of DNA demethylation process, reveals increased expression of the substrate following ES. The expression was localized in the nucleus (blue). Values are means±SEM. **p≤0.01; ***p≤0.001 by Student t test.

FIGS. 29A-B ES reduced heterochromatin configuration. (A) Graphical illustration showing the histone distancing following increased TET1 and DNA demethylation, thereby promoting translational activity. (B) Immunolabeling of H3K9me3 (green) used as a marker to reveal the distribution of histone-DNA complex in the nuclei. Note PC12 cells treated with ES after 24 hr culturing (panel 1 and 3) showed periphery localized H3K9me3 with a distinctive (panel 3 and 4) translational pattern compared to non-ES-treated cells. ES condition: 75 μA-biphasic/ramp/50 ms for 20 minutes-once.

FIGS. 30A-C Electrical Stimulation increased neurite outgrowth in mouse retinal explant cultures. (A) Immunodetection of 111-tubulin-labeled neurites (arrowhead) in representative adult mouse retinal explant cultures with and without ES. (B,C) Quantification using LASX software reveals significantly longer (B) and increased number of neurites in cultures subjected to ES compared to unstimulated cultures. After, 24 hours in culture in trans-well membranes, the tissues were subjected to ES. The images were presented in grayscale (panel 1 and 2) for optimal and clear presentation and analysis. Values are mean±SEM. *P<0.05 by Student t test.

FIGS. 31A-D Electrical stimulation alleviates RGC damage and functional deterioration in ONC model. (A) Graphical abstract of the in vivo experimental plan. (B) Representative images of immunohistochemistry of Thy1-YFP mouse retinal sections showing YFP+RGCs (green; cells in the inner most layer) bearing long nerve fibers in the ES group (arrowheads). (C) ES improves ERG responses in ONC mice. Following ONC, the pSTR values dropped significantly compared to non-injured control eye (Cnt); ES-treatment improved the ERG pSTR amplitude. Values are mean±SEM. *P<0.05; **P<0.01 by student t test (D) Retinal sections from non-ES and ES-treated Thy1-YFP transgenic mice immunostained for 5hmc. Thy1-YFP positive RGCs concomitantly expressing higher 5hmc in ES mice group, but much less seen in non-ES mouse RGCs. ONL: Outer Nuclear Layer; INL: Inner Nuclear Layer; IPL: Inner Plexiform Layer; GCL: Ganglion Cell Layer.

FIGS. 32A-F ES reduces ATP production in primary human endothelial cells. (A, B) The Fura-2 AM Ca²⁺ curve and [Ca²⁺]ᵢ quantification in ES treated cells in response to ATP 10⁻⁷ M stimulation with Ca²′ present in the KRB buffer. Statistical significance was determined by Student's t-test. ****p≤0.001; value=means±S.D. n=7 control; n=6 ES group. (C, D) Results of Agilent Seahorse oxygen consumption rate and OCR rates for Basal respiration, maximum respiration, proton leak, and ATP production in primary human endothelial cells with and without ES. Statistical significance was determined by Student's t-test. ns p>0.05; *p≤0.05; value=means±S.D. (E) Endothelial cells membrane potential in control cells and membrane potential depolarization visualized by FluoVolt after 60 min of ES. The images represent 488 nm excitation fluorescence with an applied heatmap. Quantification of FluoVolt signals over time (F). Statistical significance was determined by two-way Anova. *p≤0.05; value=means±S.D. n=3 independent experiments per time point; 20+ cells measured per field and averaged.

FIGS. 33A-B Superficial corneal stroma was removed in Thy-1-YFP mice, and TpEs was applied for 14 days with ramp waveform at 20 Hz, 300 μA. Reduced vimentin (red) and MMP-9 (blue) signal was observed in TpES cornea compared to control (right). More corneal nerves are regenerated in TpES cornea (green) and entering the central cornea (shown in white circle).

FIGS. 34A-F Superficial corneal stroma was removed in Thy-1-TYP mice, and TpES was applied for 21 days with ramp waveform at 20 Hz, 300 μA. Images of the central cornea (400×) is shown in TpES eye (A) and control (B). Greater nerve length was found in TpES cornea than control (C). Neurons were isolated from TGs of the same Thy-1-YFP mice for 24 hours, and observed under a microscope with GFP channel. The total length of the axons is significantly higher in ES-treated neurons than control (F). *p≤0.05, **p≤0.01.

FIGS. 35A-C Neurons isolated from TGs of C57BL/6J mice were cultured. ES was applied to neurons with ramp waveform at 20 Hz, 100 μA and axons were stained with β-III-tubulin (red) 24 hours after ES (A and B). The total length of the axons is significantly greater in ES-treated neurons than control (C). *p≤0.05.

FIGS. 36A-B Neurons isolated from TGs of C57BL/6J mice were cultured for 24 hours. ES was applied to neurons with ramp waveform at 20 Hz, 100 μA and [Ca²⁺]ᵢ level was measured with the presence of ATP (10⁻⁷M). The average [Ca²⁺]ᵢ over time of each preparation is shown in (A), and the [Ca²⁺]ᵢ increase from baseline is shown in (B). Arrow indicates the time point of ATP addition. ES significantly attenuated the [Ca²⁺]ᵢ increase induced by ATP. *p≤0.05.

FIG. 37A-B Neurons isolated from TGs of C57BL/6J mice were cultured. ES with sine waveform at 20 Hz, 100 μA, was applied to neurons and axons were stained with Transient receptor potential cation channel (TRP)A1 (red) and β-III-tubulin (green) 24 hours after ES. The total length of the axons is significantly greater in ES-treated neurons (A) than control (B).

FIG. 38 ES improves visual functions in I/R mice over time. The spatial vision of visual acuity (VA) and visual contrast sensitivity (CS) from Sham or ES-treated mice at 0, 1, 2, 3, and 4 weeks post-I/R injury. Data were presented as mean±SEM. Each dot represents an individual animal. *p≤0.05; **p≤0.01; ***p≤0.001 (Student's t-test).

FIG. 39 ES maintained the RGC function of pSTR in ischemia/reperfused (I/R) mice over time. Quantification of pSTR in Sham or ES-treated mice at 0, 1, 2, 3, and 4 weeks post-I/R injury after normalized to the baseline of each group. Data are presented as mean z SEM. Each dot represented an individual animal. *p≤0.05 Student's t-test.

FIG. 40 ES improved rod photoreceptor function in ischemia/reperfused (I/R) mice over time. Quantification of scotopic ERG a wave analysis in Sham or ES-treated mice at 0, 1, 2, 3, and 4 weeks post-I/R injury after normalized to the baseline of each group. Data are presented as mean±SEM. Each dot represents an individual animal. *p≤0.05 Student's t-test.

### DETAILED DESCRIPTION

### Definitions

Unless specifically defined otherwise, all technical and scientific terms used herein shall be taken to have the same meaning as commonly understood by one of ordinary skill in the art (e.g., in cell culture, molecular genetics, and biochemistry).

As used herein, the singular forms “a,” “an,” and “the” include the plural reference unless the context clearly dictates otherwise. Thus, for example, a reference to “a disease,” “a disease state”, or “a nucleic acid” is a reference to one or more such embodiments, and includes equivalents thereof known to those skilled in the art and so forth.

As used herein, the term “about” or “approximately” in the context of a numerical value or range means ±10% of the numerical value or range recited or claimed, unless the context requires a more limited range.

As used herein, “effective” when referring to an amount of a therapeutic compound refers to the quantity of the compound that is sufficient to yield a desired therapeutic response without undue adverse side effects (such as toxicity, irritation, or allergic response) commensurate with a reasonable benefit/risk ratio when used in the manner of this disclosure.

By “reference” is meant a standard or control condition.

The terms “subject.” “patient,” “individual,” and the like as used herein are not intended to be limiting and can be generally interchanged. The subject is a mammal in need of such treatment, e.g., a subject that has been diagnosed with an ocular injury or disease associated with vision loss, retinal cell damage, or retinal degeneration. The subject has an injured or diseased eye. For example, the subject suffers from photoreceptor degeneration. The mammal is, e.g., a human, a primate, a mouse, a rat, a dog, a cat, a horse, as well as livestock or animals grown for food consumption, e.g., cattle, sheep, pigs, chickens, and goats. Preferably, the mammal is a human. An individual described as a “patient” does not necessarily have a given disease, but may be merely seeking medical advice. The term “subject” as used herein includes a subject diagnosed with an optic neuropathy, for example age-related macular degeneration or photoreceptor degeneration.

As used herein, a “symptom” associated with a disorder includes any clinical or laboratory manifestation associated with the disorder, and is not limited to what the subject can feel or observe.

As used herein, “treating” encompasses, e.g., inhibition, regression, or stasis of the progression of a disorder. Treating also encompasses the amelioration of a symptom or symptoms of the disorder. As used herein, “inhibition” of disease progression or a disease complication in a subject means preventing or reducing the rate, frequence, or risk of disease progression and/or disease complications in the subject. The terms “preventing” and “prevention” refer to the administration of a therapeutic protocol to a clinically asymptomatic individual who is susceptible or predisposed to a particular adverse condition, disorder, or disease, and thus relates to reducing the risk of the occurrence of symptoms and/or their underlying cause.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

### Electrical Stimulation

In some embodiments of the methods of the disclosure, electrical stimulation is delivered to the eye. In some embodiments, the electrical stimulation is delivered to the cornea. In some embodiments, the electrical stimulation is delivered through the skin around the eye. In some embodiments, the electrical stimulation is delivered to the orbit around the eye. In some embodiments, the electrical stimulation is delivered transpalpebrally.

In some embodiments, different electrical stimulation patterns are administered. In some embodiments, the electrical stimulation is current (e.g., amps, mA, or μA) and, the current is between 1 μA and 5,000 μA (e.g., 1, 20, 50, 100, 150, 200, 250, 300, 350, 400, 450, 500, 550, 600, 650, 700, 750, 800, 850, 900, 950 1,000 μA, 1.5 mA, 2 mA, 2.5 mA, 3 mA, 3.5 mA, 4 mA, 4.5 mA, 5 mA, or any amount therein). In some embodiments, the electrical stimulation is amplitude priority stimulation. In some embodiments, the electrical stimulation required for one species (e.g., mice) may be different from another species (e.g., human).

In some embodiments, the frequency of the electrical stimulation is between 2 and 200 Hz, for example, 10 Hz, 20 Hz, 25 Hz, 30 Hz, 40 Hz, 50 Hz, 60 Hz, 70 Hz, 80 Hz, 90 Hz, 100 Hz, 110 Hz, 120 Hz, 130 Hz, 140 Hz, 150 Hz, 160 Hz, 170 Hz, 180 Hz, 190 Hz, or 200 Hz.

In some embodiments, the electrical stimulation is administered for 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 minutes a day. In some embodiments, the electrical stimulation is administered for between 2 and 1,800 seconds, in some cases 60, 100, 160, 180, 240, or 300 seconds a day. In some embodiments, the electrical stimulation is administered in multiple, divided treatment sessions throughout the day, in other embodiments, the electrical stimulation is administered in one treatment session per day.

In some embodiments of the methods disclosed herein, voltage is recorded. In some embodiments, the voltage required to reach a particular current density is measured. In some embodiments, the ramp waveform requires the least voltage to reach a target current (e.g., 100 μA, 150 μA). In some embodiments, the voltage is adjusted to target a chosen current.

In some embodiments, the electrical stimulation is a rectangular waveform. In some embodiments, the rectangular waveform is a rectangular pulse train, with varying, decreasing, or increasing frequencies from 2-200 Hz.

In some embodiments, the electrical stimulation is delivered via a ramp waveform. In some embodiments, a ramp waveform is an electric current that increases or decreases linearly with time from a predetermined negative current until it reaches a predetermined positive value or vice versa (e.g. at any absolute number between |20-2,000|μA), instantaneously returns to zero, and increases again immediately or after a predetermined time lapse as the cycle repeats. In some embodiments, the value of the current is |100|μA, |150|μA, |200|μA, |300|μA, |400|μA, |500|μA, |600|μA, |700|μA, |800|μA, |900|μA, |1000|μA, |1500|μA, or |2000|μA. In some embodiments, a ramp waveform is also called a sawtooth, or an intermittently triggered sawtooth waveform. In some embodiments, a ramp waveform is where the amplitude of a current steadily increases or decreases between two parameter amplitudes. In some embodiments, a ramp waveform ramps upward and then sharply drops. In some embodiments, a ramp waveform ramps downward and then sharply increases. Some parts of the ramp waveform may contain a direct flow of current, and some parts of the ramp waveform may contain a reversed flow of current. In some embodiments, the ramp waveform is at a pulse duration of between 0.5 millisecond to 500 millisecond, and a frequency between 2 and 200 Hz, for example 20 Hz. In some embodiments, the ramp waveform is administered for between 2 and 1,800 seconds, in some cases 60, 100, 160, 180, 240, or 300 seconds. In some embodiments, the pulse duration of a ramp waveform is between 0.5 ms and 500 ms, in some cases 50 ms. In some embodiments, the ramp waveform is divided into two phases, for example a 100 ms ramp waveform can be divided into one 50 ms negative and one 50 ms positive ramp waveform phases. In some embodiments, the electrical stimulation is a rectangular waveform followed by a ramp waveform, or a ramp waveform followed by a rectangular waveform.

In some embodiments, the electrical stimulation is a sine waveform. A sine waveform, sometimes also called a sinusoidal wave or sinusoid, is a continuous wave that oscillates periodically, and is defined by the function y=sin x. A sine wave can be depicted as two semi-circular curves that alternate above and below a center line.

In some embodiments, electrical stimulation is delivered to an eye or around the eye of a subject every day. In some embodiments, the electrical stimulation is delivered to an eye or around the eye or a subject every other day. In some embodiments, electrical stimulation is delivered nearly every day. In some embodiments, electrical stimulation is delivered most days, over half the days, or some days per week. In some embodiments, electrical stimulation is delivered every other day, every 3 days, once a week, three days on/one or two days off, four days on/one or two days off, five days on/one or two days off, six days on/one or two days off, or any other administration protocol that achieves a therapeutic benefit.

In some embodiments, electrical stimulation is delivered for at least one minute, at least two minutes, at least three minutes, at least four minutes, or at least five minutes per day. In some embodiments, electrical stimulation is delivered for no more than five minutes, four minutes, three minutes, two minutes, or one minute per day.

### Transpalpebral Stimulation

In some embodiments of the methods of the disclosure, electrical stimulation is transpalpebral (eyelid) stimulation. In some embodiments, electrical stimulation is delivered to the eye through the skin around the eye. In some embodiments, electrical stimulation is delivered to the eye through the eyelid. In some embodiments, electrical stimulation is delivered to the eye through the eyebrow. In some embodiments, the electrical stimulation is delivered within the orbit around the eye. In some embodiments, the electrical stimulation is delivered on or around the orbit around the eye. In some embodiments, transpalpebral or around the eye can include 2 cm around the eye, 3 cm around the eye, or 4 cm around the eye in any direction.

### Disease or Injury to the Eye

### Age-Related Macular Degeneration

AMD is a complex disease that involves multiple pathological processes, including chronic inflammation, oxidative stress, and abnormal angiogenesis. Current treatments for AMD are limited to slowing down the progression of the disease and managing symptoms. Electric stimulation as a therapy for AMD involves applying electrical currents to the retina to stimulate the cells and modulate their function. Electric stimulation (ES) can regulate microglia activation, which plays a critical role in the inflammatory response in AMD. Microglia are immune cells in the retina that respond to injury or inflammation by releasing pro-inflammatory cytokines and promoting the recruitment of other immune cells. In AMD, microglia are activated and contribute to the progression of the disease by releasing toxic molecules and promoting the formation of abnormal blood vessels, possibly through proinflammatory activation and motility through modulation of C^{a} and mitochondrial polarization. Microglia activation and subsequent angiogenesis is a common pathology in choroidal neovascularization (CNV), such as that occurs in the wet form of AMD or induced by ocular laser injury. Furthermore, a similar response to microcurrent ES is also observed in retinal endothelial cells. ES can negatively regulate the angiogenic potential of endothelial cells, regulating the inflammation and angiogenesis in the eye non-invasively. Delivery of therapeutic agents to the eye, such as anti-inflammatory drugs and anti-VEGF antibodies to treat laser injury, has limited efficacy and requires intravitreal injections making non-invasive microcurrent particularly appealing. In some embodiments, ES as described herein can be used to treat AMD.

### Corneal Nerve Regeneration

The cornea is heavily innervated by sensory nerve endings originated from the trigeminal ganglia (TG). Normal corneal innervation is crucial for corneal health. Conditions that potentially damage the corneal nerve include corneal refractory surgery, herpes simplex virus (HSV) infection, and diabetes mellitus. This damage to the integrity of the corneal nerve leads to decreased blink reflex, insufficient tear production, and/or ulceration. Long term effects of these symptoms can consequently lead to diminished corneal sensitivity, epithelial defects, and possible blindness. The neuro-regenerative effect of ES on limbal nerves has been well-documented in both animal models and humans. Non-invasive transpalpebral ES (TpES) presents a therapeutic approach for promoting corneal wound healing and stimulating nerve regeneration. In some embodiments, ES as described herein can be used to regenerate the corneal nerve.

### Ischemic Optic Neuropathy (ION)

Ischemic optic neuropathy (ION) is a condition that occurs when the retina and/or optic nerve, which are responsible for carrying visual information from the eye to the brain, are damaged due to a lack of blood flow. Symptoms of ION may include sudden vision loss, blurring or dimming of vision, or a decrease in visual acuity in one or both eyes permanently. To date, there is no effective treatment to improve vision loss from ION once it occurs. Electrical stimulation (ES) can have neuroprotective effects for promoting retinal and visual functions after vision loss from ION. In some embodiments, ES as described herein can be used to treat ischemic optic neuropathy (ION), including non-arteritic anterior ischemic optic neuropathy.

### Other Diseases or Injuries to the Eye

In other embodiments, the electrical stimulation protocols described herein can be used to treat other diseases or injuries to the eye including, but not limited to, ischemic retinopathy, diabetic retinopathy, traumatic optic neuropathy, glaucoma, corneal abrasions, corneal ulcers, dry eye syndrome, corneal neuropathic pain, neuropathic keratitis, and/or corneal vascularization. Electrical stimulation (ES) can have neuroprotective effects for promoting retinal and visual functions after these other diseases or injuries to the eye.

### Assessments

In some embodiments, the outcomes and/or benefits of the treatment can be assessed non-invasively using fundus scope, fluorescence angiogram, and/or optical coherence tomography (OCT). OCT is commonly used to determine eye and retinal morphology, retinal architecture, and the thickness of various layers of the retina, retinal pigment epithelia, and sclera.

In some embodiments, the methods of the disclosure can be assessed using ERG. Electroretinography (ERG) analysis is a method known in the art for assessing photoreceptor function and neural responses.

In some embodiments, the methods of the disclosure can be assessed using visual acuity, best corrected visual acuity, visual contrast sensitivity, visual field, optomotor response (OMR), and optic kinetic reflect (OKR). OMR or OKR is frequently used as a metric of visual performance.

In some embodiments, the methods of the disclosure can be assessed by measuring photoreceptor survival. Photoreceptor survival can be assessed by methods disclosed in the art.

The function of retinal ganglion cells (RGCs) can be non-invasively assessed in experimental and genetic models of glaucoma by means of variants of the ERG technique that emphasize the activity of inner retina neurons. The best understood technique is the Pattern Electroretinogram (PERG) in response to contrast-reversing gratings or checkerboards, which selectively depends on the presence of functional RGCs. In glaucoma models, the PERG can be altered before histological loss of RGCs; PERG alterations may be either reversed with moderate IOP lowering or exacerbated with moderate IOP elevation.

Under particular luminance-stimulus conditions, the Flash-ERG displays components that may reflect electrical activity originating in the proximal retina and be altered in some experimental glaucoma models (positive Scotopic Threshold response, pSTR; negative Scotopic Threshold Response, nSTR; Photopic Negative Response, PhNR; Oscillatory Potentials, OPs; multifocal ERG, mfERG) (Vittorio Porciatti, Exp Eye Res. 2015 December; 141: 164-170).

In some embodiments, preserving visual function is maintaining a similar level or amount of an outcome after identification of eye disease or injury and treatment with ES as compared to the level or amount of an outcome of a non-diseased or non-injured eye. In some embodiments, preserving visual function is maintaining a higher level or amount of an outcome after identification of eye disease or injury and treatment with ES as compared to the level or amount of an outcome after identification of eye disease or injury but without treatment with ES. In some embodiments, preserving visual function is reducing the rate of loss of a level or amount of an outcome after identification of eye disease or injury and treatment with ES as compared to the level or amount of an outcome after identification of eye disease or injury but without treatment with ES.

### Benefits

In some embodiments, the methods of the disclosure provide one or more benefits to the subject having macular degeneration, age-related macular degeneration, photoreceptor degeneration, optic nerve or retinal trauma, ischemic retinopathy, diabetic retinopathy, traumatic optic neuropathy, glaucoma, corneal abrasions, corneal ulcers, dry eye syndrome. corneal neuropathic pain, neuropathic keratitis, and/or corneal vascularization. In some embodiments, the methods of the disclosure can be used to treat macular degeneration, age-related macular degeneration, photoreceptor degeneration, optic nerve or retinal trauma, ischemic retinopathy, diabetic retinopathy, traumatic optic neuropathy, glaucoma, corneal abrasions, corneal ulcers, dry eye syndrome. corneal neuropathic pain, neuropathic keratitis, and/or corneal vascularization. In some embodiments, the method of the disclosure can be used with other methods to treat ocular disorders, for example eye drops or injections. The methods disclosed herein are compatible with other common treatments for MD/AMD or photoreceptor degeneration and are not contraindicated for those already undergoing MD/AMD or photoreceptor degeneration treatment. In some embodiments, the methods of the disclosure can promote the regeneration of photoreceptors. In some embodiments, the methods disclosed herein protect neurons and promote neuroplasticity. In some embodiments, the methods disclosed herein suppress immune response a neuroinflammation. In some embodiments, the methods disclosed herein reduce or suppress microglia activation. In some embodiments, the methods disclosed herein promote wound healing. In some embodiments, the methods disclosed herein promote nerve self-repair or growth. In some embodiments, the ramp waveform disclosed herein uses less output voltage to reach a targeted value of 100 μA current density than other waveforms (rectangular or sine waveforms).

### Abbreviations

| | |
|---|---|
| AKT | protein kinase B/PKB |
| AMD | age-related macular degeneration |
| BDNF | brain-derived neurotrophic factor |
| B-opsins | blue-opsins |
| [Ca²⁺]i | intracellular Ca²⁺ |
| CNTF | ciliary neurotrophic factor |
| CNV | Choroidal neovascularization |
| CSF | corneal fluorescein staining |
| EC | endothelial cells |
| ERG | electroretinogram |
| ES | electrical stimulation |
| HREC | human retinal endothelial cells |
| MD | Macular degeneration |
| mTOR | mammalian target of rapamycin |
| MUC | mucin |
| PI3K | phosphoinositide 3-kinase |
| PIP3 | phosphatidylinositol 3,4,5-triphosphate |
| PTEN | phosphatase and tensin homolog |
| OCT | optical coherence tomography |
| OMR | optomotor response |
| ONL | outer nuclear layer |
| RCSC | RPE/Choroidal/Scleral |
| RGC | Retinal ganglion cell |
| RP | retinis pigmentosa |
| R/G-opsins | red/Green-opsins+\ |
| Rho−/− | rhodopsin knockout |
| RPGR | RP caused by mutations in the GTPase regulator |
| tES | transcorneal electrical stimulation |
| TcES | transcorneal electrical stimulation |
| TpES | transpalpebral electrical stimulation |
| VA | visual acuity |
| ZO-1 | zonular occludens |

### EXAMPLES

The invention is further described in the following examples, which do not limit the scope of the invention described in the claims.

### Example 1 Current Waveforms and Electrical Field Penetration into the Posterior Segment of the Eye

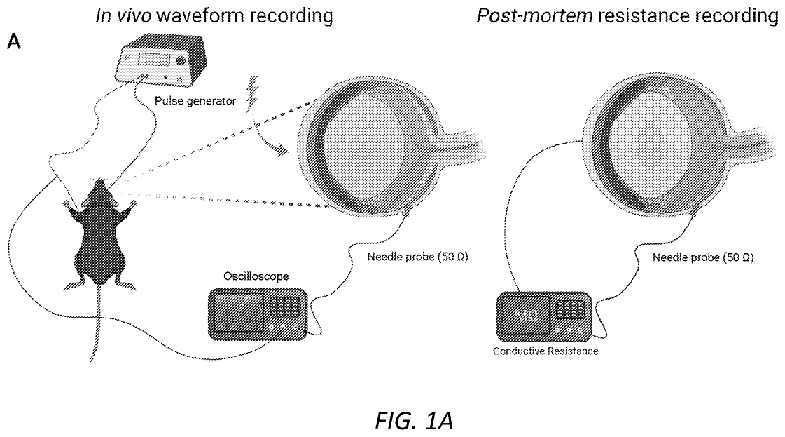

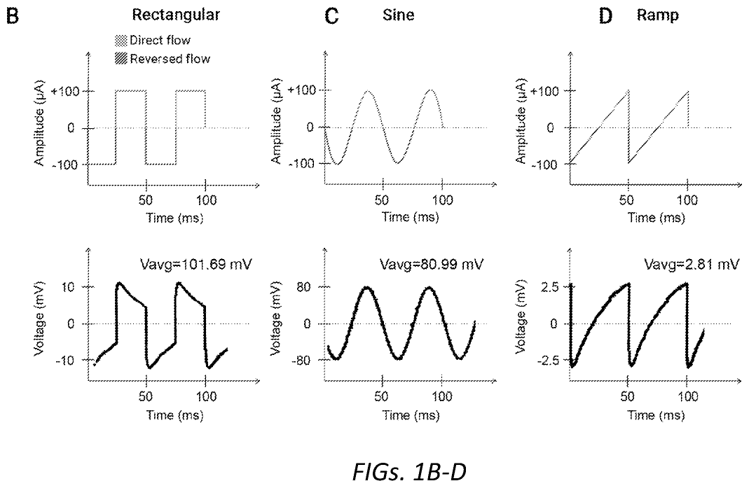

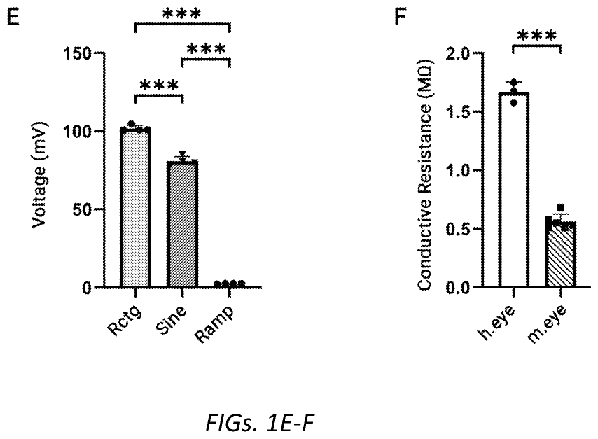

We recorded the current waveforms and electric field densities in the subretinal space using an oscilloscope Hantek2000 (Gyeonggi, Republic of Korea) for electrical stimulation (ES) waveform and voltage recordings to test if transcorneal ES (tES) reaches the posterior eye or retina. The needle probe (50Ω) of the oscilloscope was inserted into the subretinal space of an anesthetized mouse (FIG. 1A, left). ES at a biphasic rectangular, sine or ramp waveform was delivered at 20 Hz, a frequency previously shown to be optimal for retinal cells. The corresponding output waveform was recorded following all ES (FIG. 1B-D). Intriguingly, the output voltage needed to reach a targeted value of 100 μA current density in the subretinal space of anesthetized mice varied significantly among different waveforms. On average, biphasic rectangular required 101.69 mV, and sine was 80.99 mV, while ramp waveform used only 2.81 mV (FIG. 1E). The results suggest that the ramp waveform is most efficient in delivering the electrical current and requires the least voltage potential to reach the retina.

To test if the ES current flow assessed in the mouse eye can be translated to humans, we compared the ES-induced currents and conductive resistance in the mouse and donor human eyes. The conductive resistance of post-mortem mouse and human eyes was measured by placing multimeter probes on the cornea and a needle in the subretinal space (FIG. 1A, right). The transcorneal-subretinal resistance measured from the post-mortem human eye was averaged to 1.66±0.07 MΩ, which was 3× higher than that measured in the post-mortem mouse eye, where the conductive resistance values were 0.56±0.05 MΩ (FIG. 1F). These data suggest that approximate 3× higher voltage potentials are required to achieve the targeted amplitude in the human eyes by ES than the mouse eyes because of the higher tissue resistance.

### Example 2 Electric Stimulation Ameliorates Inflammation and Angiogenesis in Mouse Laser CNV Model

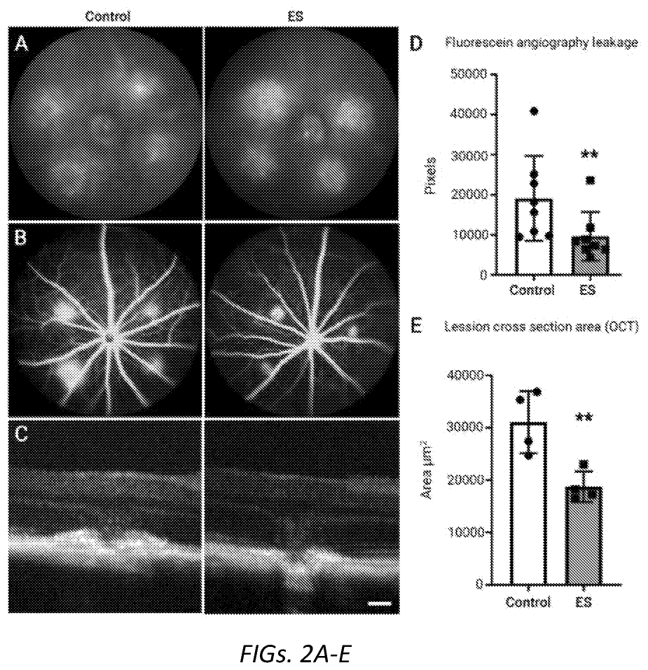

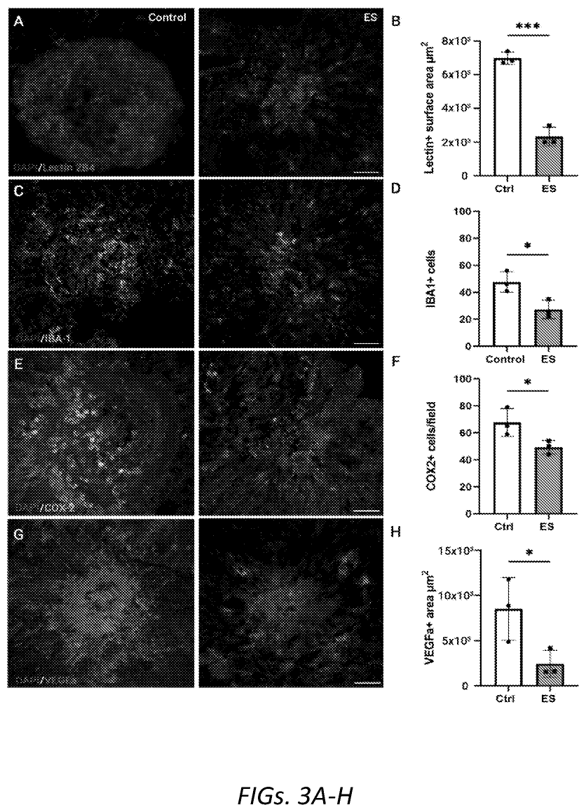

To assess the therapeutic potential of transpalbabral ES on laser eye injury, adult C57BL6 mice were subjected to laser damage at the 9, 12, and 3 o'clock positions of the posterior pole of the retina with the Micron IV equipped with laser injector system OcuLight GL diode laser (532 nm wavelength, Index, Mountain View, CA, USA). The transpalbabral ES was performed with STG4000 (Multichannel Systems, Reutlingen, Germany) pulse generator, the ES probe was applied to the skin of the orbit and the negative electrode to the abdominal area of the mice. TpES was applied with ramp waveform at 20 Hz, 300 uA to the injured eye for 4 min a day for 7 days. The contralateral eyes that received sham treatment without delivering an ES were used as controls (FIG. 2). The in vivo fluorescent angiography (FA) and OCT evaluation of the laser spot size indicate a significant reduction of the fluorescein leakage (FIG. 2B, D). Optical coherence tomography (OCT) results further verified reduced retinal damage in ES-treated eyes compared to controls (FIG. 2C, E). We next investigated the angiogenesis, inflammatory cell infiltration, and VEGF expression within the laser CNV spots by isolating RPE/Choroidal/Scleral (RCSC) complexes of the sham and ES-treated animals and incubating with lectin (Lycopersicon esculentum lectin; FIG. 3 A, B) to visualize the endothelial cells or primary antibodies IBA-1 (1:100; 019-19741; FIG. 3C, D), COX-2 (1:100; MA5-14568; FIG. 3 E, F) or VEGFa (sc-7269; FIG. 3 G, H). The immunostaining of the RCSC and area quantification of lectin-positive signals indicates a significant reduction in lectin-positive endothelial cells (FIG. 3A, B) as well as reduced infiltration of IBA-1-positive inflammatory cells into the laser CNV spot (FIG. 3C, D), reduced expression of COX-2 (FIG. 3 E, F) and VEGFa (FIG. 3 G, H). This data indicates the anti-inflammatory and anti-angiogenic effect of the ES in ocular laser injury.

### Example 3 ES Reduces Microglia Activation by Modulation of Intracellular Ca²⁺ In Vitro

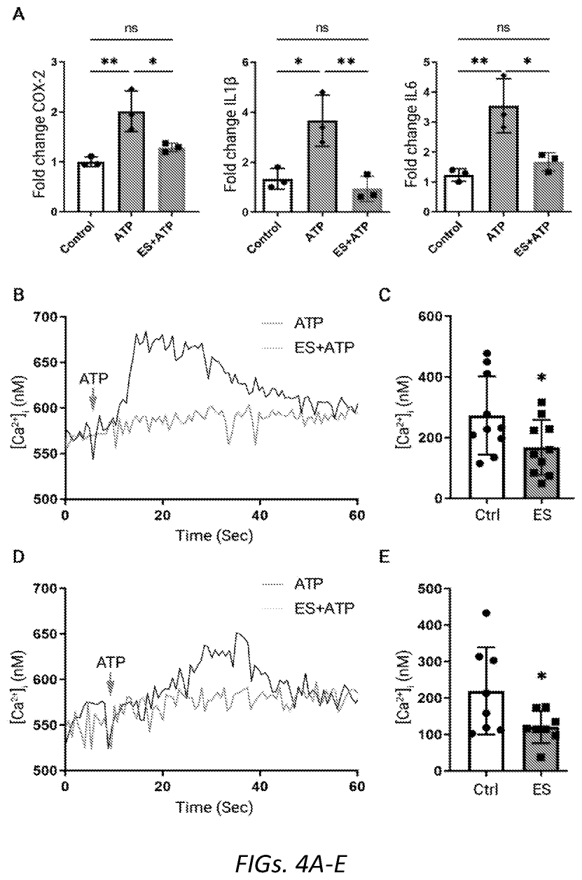

We followed up these existing in vivo results with in vitro investigation of the underlying mechanism of the ameliorative effects by microcurrent stimulation. We isolated microglia cells from adult C57BL/6 (WT) mice brains by papain dissociation according to the protocol published in the literature. The flow cytometry quantification using IBA1 immunolabeling demonstrated above 98% purity of IBA1+ cells in cultures (data not shown). Cells were maintained in Dulbecco's modified Eagle's medium (DMEM) that was supplemented with 10% fetal bovine serum (FBS) and 1% penicillin/streptomycin and 50 ng/ml of Recombinant Mouse (rm) Colony Stimulation Factor 1 (CSF1) (416-ML). The ES in cultures was performed with STG4000 (Multichannel Systems, Reutlingen, Germany) pulse generator using: biphasic ramp waveform (300 μAmp, 20 Hz, 1 h). The electric current was delivered to cultures using a c-dish carbon electrode plate (Ion Optix, Westwood, MA, USA). The most characteristic feature of microglia is their rapid activation in response to acute pathological events, including cell death, neurodegeneration, and inflammation in the retina. Microglia possess functional receptors that recognize Adenosine triphosphate (ATP) released from the dead cells, i.e., GTP-binding protein (G-protein)-coupled P2 receptors such as P2Y2, P2Y12-like receptor, and ionotropic P2 receptors such as P2X7 20 ATP evokes electrical currents in microglia-mediated by intracellular calcium ([Ca²⁺]ᵢ) spike in mouse and human microglia. To mimic an inflammatory insult consistent with retinal laser injury in vitro, we used the ATP 10-M added to the microglia cultures and incubated for 12 hours. The expression of COX-2, IL-6, and IL-1β major microglia proinflammatory activation markers were investigated by RT-PCR (FIG. 4A). The ES significantly downregulated the transcription of each of COX-2, IL-1β, and IL-6 (FIG. 4A). As microglia activation highly depends on the [Ca²⁺]ᵢ currents spike, we investigated [Ca²⁺]ᵢ activity in control and ES-treated microglia cells. The primary microglia cells were seeded onto 35-mm glass-bottom culture dishes and incubated at 37° C., 5% CO2 overnight. Microglia cultures were subjected to microcurrent ES as described above. Cells were then incubated for 1 h at 37° C. with Krebs-Ringer bicarbonate buffer (KRB) containing 119 mM NaCl, 4.8 mM KCl, 1.0 mM CaCl2, 1.2 mM MgSO4, and 25 mM NaHCO₃ with 4-(2-hydroxyethyl)-1-piperazineethanesulfonic acid (HEPES) plus 0.5% BSA containing 0.5 M fura-2/AM (Invitrogen, Grand Island, NY, USA), 8 M pluronic acid F127 (Sigma-Aldrich, St. Louis, MO, USA) and 250 μM sulfinpyrazone (Sigma-Aldrich) for 1 h. Before calcium measurements were started, cells were washed with KRB-HEPES containing sulfinpyrazone. Ca²⁺ measurements were conducted using a ratio imaging system (In Cyt Im2; Intracellular Imaging) using wavelengths of 340 and 380 nm and an emission wavelength of 505 nm. Cells were stimulated with ATP 10⁻⁷. The intracellular Ca²⁺ ([Ca²⁺]ᵢ) over time was recorded, and the change in peak [Ca²⁺]ᵢ was calculated by subtracting the average of the basal value from the peak [Ca²⁺]ᵢ value. The ES has significantly reduced the ATP invoked [Ca²⁺]ᵢ peak, (FIG. 4B, C), further confirming the anti-inflammatory effect of the ES on microglia cells.

To understand if ES disrupts the calcium uptake by the microglia from the extracellular space or if ES mediates intracellular Ca²⁺ sources, we modified the Ringer bicarbonate buffer (120 mM NaCl, 4.8 mM KCl, 1.2 mM MgSO4, and 25 mM NaHCO₃ with HEPES and 0.5% BSA containing 0.5 μM fura-2/AM to exclude any extracellular source of calcium. Similar results were obtained in the subsequent experiments to determine the elevation of [Ca²⁺]ᵢ in microglia in response to ATP stimulation with no sources of extracellular Ca¹. (FIG. 4D, E). These data indicate that the ES modulates calcium depolarization from intracellular stores rather than acting through transmembrane calcium channels.

### Example 4 Electric Stimulation Reduces Mitochondrial Polarization

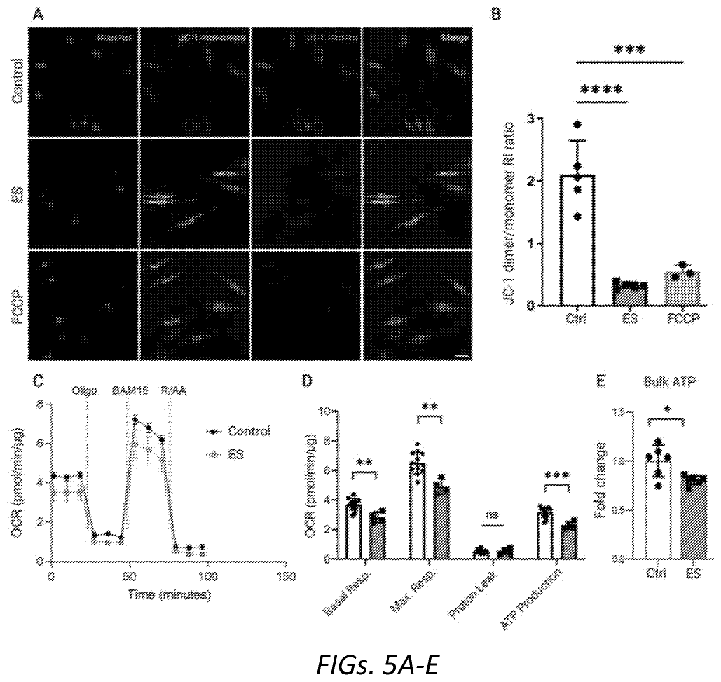

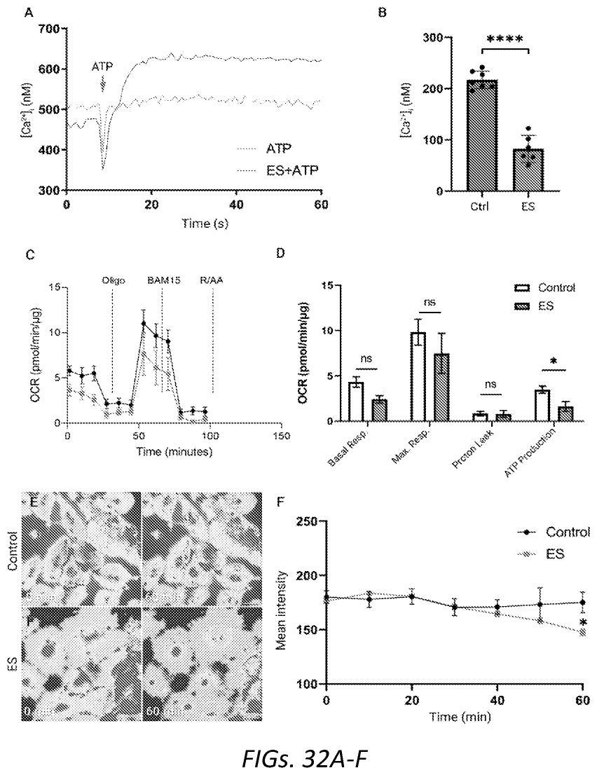

The intracellular calcium is predominantly stored in the endoplasmic reticulum (ER), which is dependent on a constant supply of ATP from mitochondria to maintain its calcium reserve. It is likely that ES reversibly depolarizes mitochondrial membranes resulting in reduced cellular metabolism and, as a result, depletion of the calcium ER depolarization. To confirm it, we investigated the effects of ES on mitochondrial membrane potential using JC-1 staining T3168. JC-1 is the cationic dye that exhibits potential-dependent accumulation in mitochondria, indicated by a fluorescence emission shift from green (˜525 nm) to red (˜590 nm). Consequently, mitochondrial depolarization is indicated by a decrease in the red/green fluorescence intensity ratio. The potential-sensitive color shift is due to the concentration-dependent formation of red fluorescent J-aggregates. The green-to-red fluorescence ratios depend only on the membrane potential and not on other factors such as mitochondrial size, shape, and density. The primary microglia cells were subject to ES and loaded with 10 μg/ml of JC-1 dye. Unstimulated cells and Carbonyl cyanide-p-trifluoromethoxyphenylhydrazone (FCCP), mitochondrial oxidative phosphorylation uncoupler (ab120081) treated cells were used as the negative and positive control, respectively. We observed mitochondrial depolarization followed by ES, similar to FCCP control (FIG. 5A). Quantitative analysis of JC-1 dimers to monomer ratios indicated a significant decrease in mitochondria polarization induced by the ES (FIG. 5B). We have followed up these experiments with high-resolution respirometry. The Seahorse XFe96 Analyzer (Agilent Technologies, Santa Clara, CA, United States) was used to determine real-time bioenergetic profiles. Fifty thousand primary microglia cells were seeded per well, incubated overnight at 37 C, 5% CO2 and subjected to ES followed by the Mito Stress Test. The medium was replaced with the assay medium (Seahorse XF Base Medium without Phenol Red, Agilent) supplemented with 2 mM GlutaMAX (ThermoFisher), 1 mM pyruvate (Gibco, Carlsbad, CA, United States) and 25 mM glucose (Sigma, St. Louis, MO, United States), pH 7.4 and placed in a 37 C humidified incubator for 1 h. The drug injections were oligomycin (2.5 mM), BAM15 (10 mM) and a combination of rotenone and antimycin A (both at 2 mM). On completion of the Seahorse assays, cells were lysed in a cold Cell Lysis Buffer (Cell Signaling Technology, Beverly, MA, United States) supplemented with 1 mM PMSF (Sigma, St Louis, MO, United States). Protein concentration was quantified using the Pierce BCA Assay kit (ThermoFisher, Waltham, MA, United States). Data were normalized to protein content. The electric stimulation significantly reduced the oxygen consumption rate curve (OCR) (FIG. 5C). The respirometry analysis indicated a significant reduction of basal cellular respiration and maximum respiration. However, the proton leak parameter indicating irreversible mitochondrial damage leading to cell death was not significant, signifying the relative safety of the biphasic ramp current effects and the reversible nature of mitochondrial depolarization. As expected from JC-1 staining experiments, the ATP production was also significantly decreased (FIG. 5D). The respirometry results were further confirmed by direct quantification of intracellular ATP content using the ATP bioluminescence assay kit CLS II (Roche, Heidelberg, Germany), based on the light-emitting oxidation of luciferin by luciferase in the presence of ATP. Consistent with respirometry data, the bulk ATP content of the microglia cells was significantly decreased by the ES (FIG. 5E). Further, ES affects the angiogenic potential of human retinal endothelial cells (HREC), which play a crucial role in the formation of laser-induced choroidal neovascularization (CNV). The results of intracellular calcium and high-resolution respirometry in primary human endothelial cells were similar to those observed in microglia cells (FIGS. 32A-F).

### Example 5 Electric Stimulation Induces an Intracellular Release of Positive Ions Decreasing Cellular Polarization Consistent with Endoplasmic Reticulum Ca²⁺ Depolarization Depletion

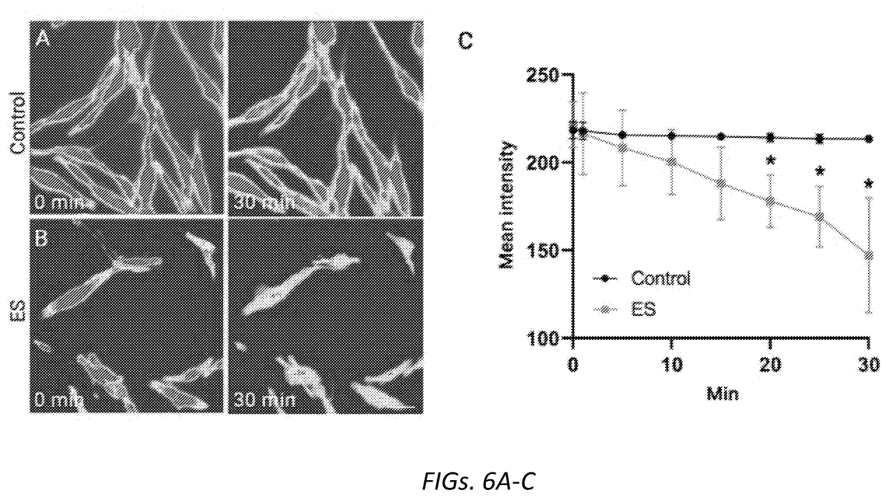

To indirectly confirm the depletion of ER calcium depolarization by the ES, we utilized a novel FluoVolt membrane potential kit (F10488) which produces 488 nm fluorescence based on cellular membrane potential. We predicted that under the effects of the ES, we would observe a gradual decrease of cellular membrane potential due to the release of positive ions from the ER. The cells were loaded with FlouVolt 1 μM and observed in time-lapse images with initial observation at 1 min, followed by image series at 5 min intervals, during the ES. The control cells were imaged with the c-plate in place but without applying an electric current. Sham-stimulated microglia did not indicate substantial changes in cellular polarization (FIG. 6A), however, applied microcurrent ES demonstrated a progressive decrease of microglia membrane potential consistent with the intracellular release of positive ions such as Ca²⁺ (FIG. 6B). Quantitative analysis of the mean fluorescence intensities of the cells indicated a progressive decrease of cellular membrane potential that became apparent at 10 min and significant at 20 min (FIG. 6C).

### Example 6 Electric Stimulation Reduces the Angiogenic Potential of Human Retinal Endothelial Cells

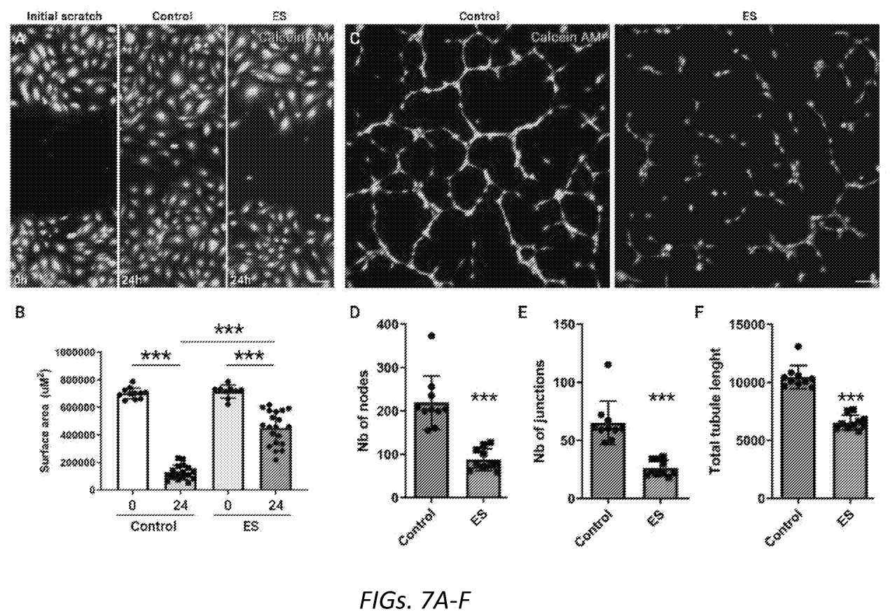

Migration and proliferation of endothelial cells (EC) is another critical component of laser CNV formation. EC depends on the Ca²⁺ flux event that triggers increased VEGF production and angiogenesis. Thus, we investigated whether the effects observed in microglia translate to the EC angiogenic properties in vitro using human retinal endothelial cells (HREC). HREC cultures were purchased from Cell System (ACBRI 181) and cultured in an EGM-2 media with bullet kit supplementation (Lonza). First, we confirmed the effects of the ES on the migration capacity of EC using the scratch assay. HREC were grown in a 6-well-plate, and the migration assay was conducted in a confluent HREC monolayer. A scratch along the diameter of the well was introduced with the 1 ml pipet tip with a gap distance of approximately 1 mm. Then, cultures were stimulated with biphasic ES ramp waveform at 100 μA, 20 Hz for 1 h. Cells were visualized by staining with Calcein AM 1 μg/ml (C3100MP), and the surface area of the gap was measured at 0 and 24 hours by the “masked” observer using ImageJ software. The ES has markedly delayed the scratch closure (FIG. 7A). The quantitative analysis of the surface area in control and ES cultures indicated a significantly higher surface area in the ES cultures (FIG. 7B).

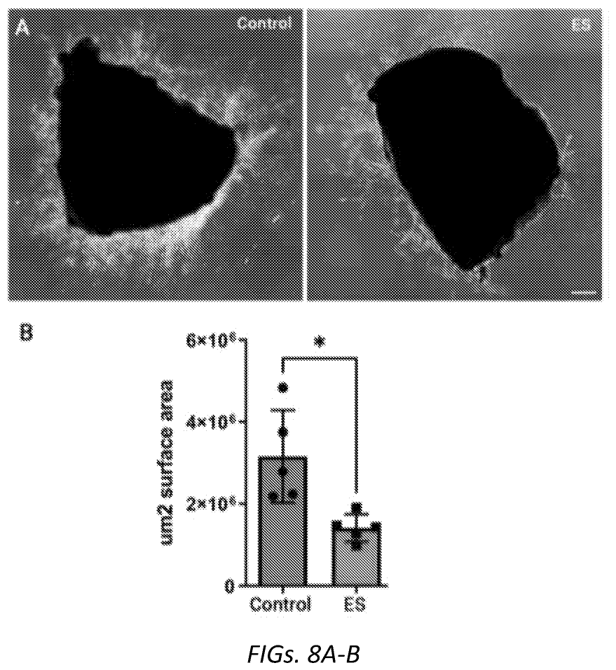

Next, we used tube formation assay as an easy and reliable method to evaluate the angiogenic potential of the HREC under the effects of microcurrent. 5×105 HREC were seeded onto a matrigel pre-coated 6-well-plate and subjected to ES. Sham-stimulated HREC was used as a control. Cultures were then incubated for 12 hours, and Calcein AM visualized tubular network formation. The images revealed substantial disruption of tubular network formation in the ES-treated cultures (FIG. 7C). Quantification of the number of nodes, the number of junctions and total tubule length was performed by Angiogenesis Analyzer and indicated a significant reduction of nodes (FIG. 7D), junctions (FIG. 7E) and total tubule length (FIG. 7F). To confirm the HREC in vitro results, we performed RPE/Choroidal explant outgrowth assay. The C57BL6 mouse eyes were enucleated and dissected under the microscope; retinas were removed to produce an RPE/Choroid eye cup. The cup was cut into 4 segments ˜2 mm in diameter, then placed in a matrigel droplet in a 6-well plate, cultured in EGM-2 media with bullet kit supplementation (Lonza) and subjected to ES daily. The endothelial cells' outgrowth from the explant was quantified after 7 days of cultures. The area of the outgrowth was determined by image J, by quantifying the total surface area of the explant and outgrowth and subtracting the surface area of the explant. The daily electric stimulation has significantly reduced the explant cellular outgrowth (FIG. 8A, B).

### Example 7 Electric Stimulation Reduces VEGFa Expression in Endothelial Cells

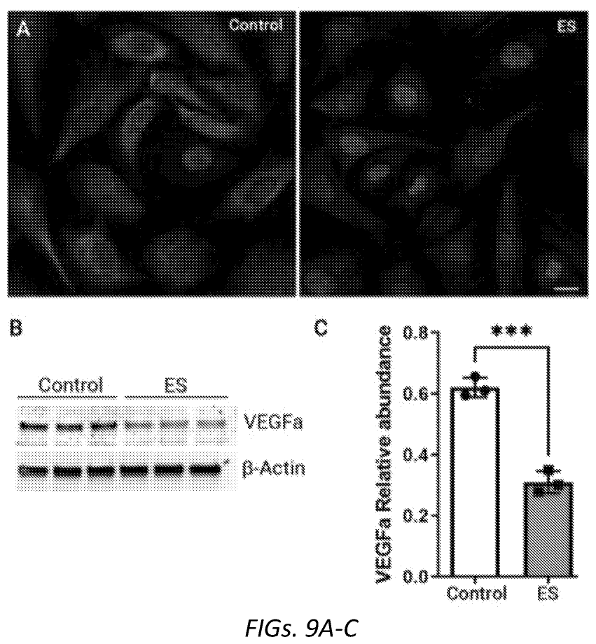

The observed anti-angiogenic effect of the biphasic ramp was suggestive of reduced angiogenic potential of endothelial cells. The VEGF is the major angiogenesis factor, and the increased calcium currents drive its expression in EC. We investigated the effects of ES on VEGF expression by subjecting HREC to ES and performing immunostaining in the fixed EC and western blotting analysis to VEGFa. Briefly, the HREC were cultured and subjected to ES as described above. Cells were fixed and immunolabeled with antibodies to the VEGFa (sc-7269). Other cultures were lysed, and lysates were separated by SDS-PAGE gel electrophoresis and transferred to a nitrocellulose membrane. The sc-7269 antibody detected the expression of VEGFa, and β-actin (MA5-15739) was used as the loading control. Densitometry was performed using ImageJ. In both immunolabeling (FIG. 9A) and western blotting results (FIG. 9B), the microcurrent ES demonstrated reduced expression of VEGFa by HREC. The densitometry analysis confirmed a significant reduction of VEGF expression by the HREC (FIG. 9C). Combined these results suggest the exciting possibility of using non-invasive transpalbabral ES as a novel bioelectric form of ocular therapy that reduces inflammatory activation of the endothelial cells and angiogenic potential of endothelial cells.

### Example 8 TcES Induces Dry Eye after 14 Days of Continuous Application in Mice

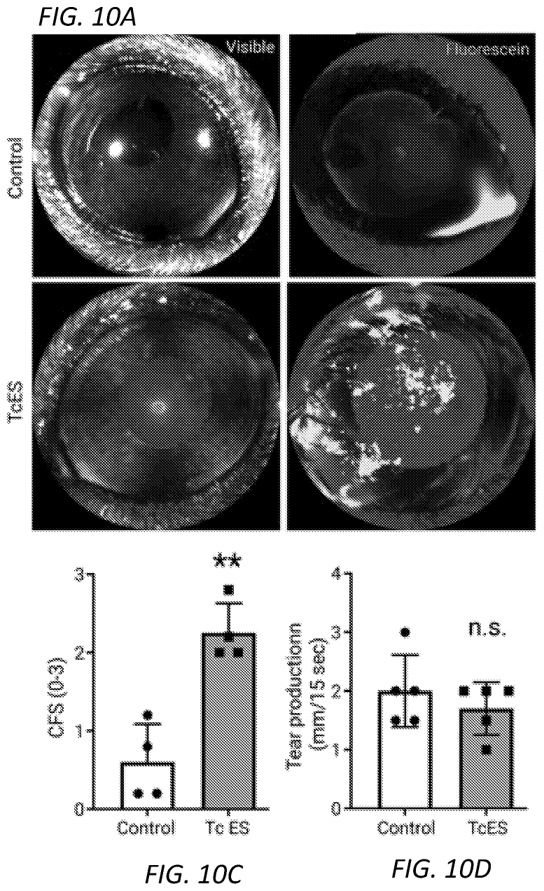

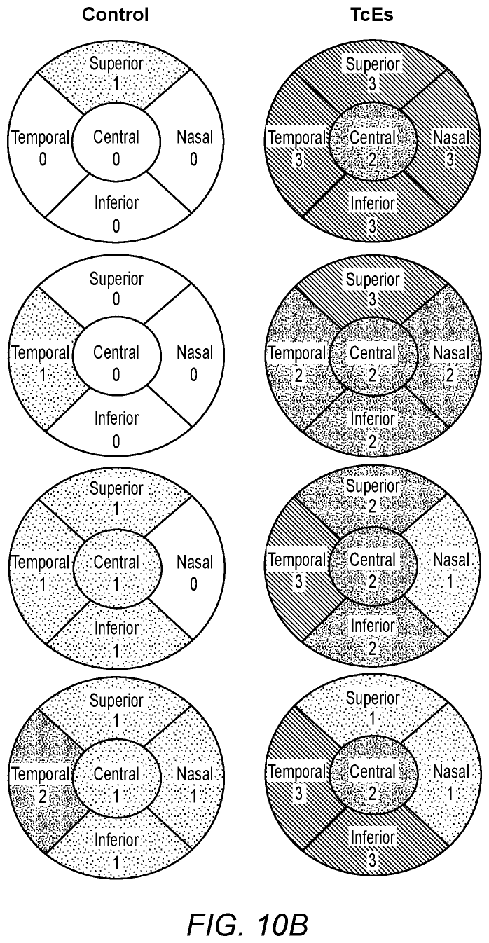

First to determine whether TcES can induce dry eye in mice, the right eye of each animal underwent a short TcES session of 4 min per day for 14 consecutive days through a conductive gel. The same conductive gel and electric probe was applied to the left eye without ES as vehicle control. On day 14, a corneal fluorescein staining test was performed to assess ocular surface damage. The slit lamp observation showed severe corneal fluorescein staining in the TcES eye, while minimum to no staining in the contralateral eyes (FIGS. 10A and B). Quantification data revealed significantly increased fluorescein staining score in the TcES eyes compared to the control eyes (p=0.0018, FIG. 10C), implicating the induction of dry eye.

We next determined whether the dry eye is due to the lack 299 of tear production (aqueous deficient). Phenol red thread was inserted into the lower conjunctival sac of the mouse eyes after 14 consecutive days of TcES or sham-treatment, and the length of the color change section was measured to indicate the tear production rate of the measured eye. No significance difference was found in the tear production rate between TcES eye and the control (p=0.40, FIG. 10D). This result suggests that the dry eye caused by TcES is not due to aqueous deficiency but may be caused by the disturbance of other components of the tear film.

### Example 9 TcES Decreases Transmembrane Mucin Production of the Corneal Epithelium In Vivo

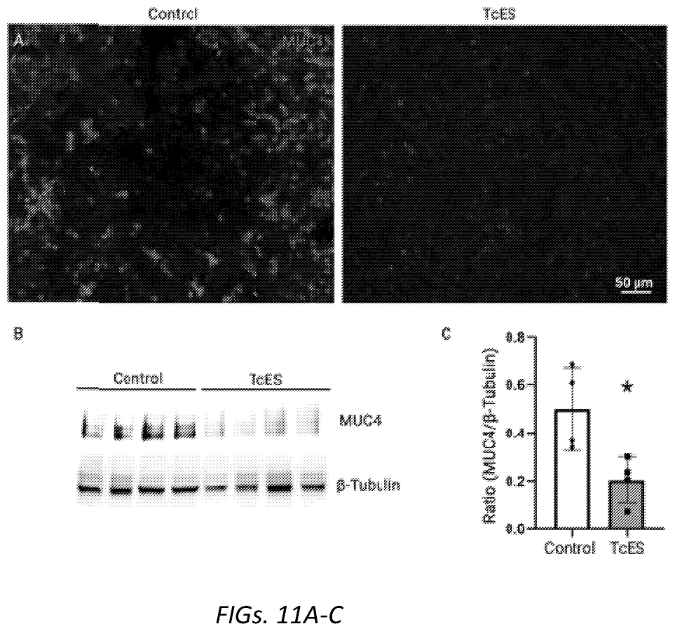

To investigate the mechanism of the TcES-induced corneal epithelial damage, we examined mucin production in the tear film. Both TcES and control eyes were collected from the animals after 14 days of ES, and corneal whole-mount staining was performed using primary antibodies against MUC4, which is commonly expressed by both human and mouse corneal epithelial cells. The immunoreactivity of MUC4 was present on the apical layers of the corneal epithelium in control eyes but was markedly reduced in TcES eyes (FIG. 11A). This was confirmed by Western blot analysis using corneal tissues treated with TcES and the untreated contralateral eyes (FIG. 11B). Quantification results showed a near 60% decrease in MUC4 protein signal in the TcES group compared to the contralateral eye (p=0.025, FIG. 11C), indicative of TcES-induced corneal MUC4 deficiency.

### Example 10 Electrical Stimulation Directly Disturbs Transmembrane Mucin Production and the Tight Junctions of the Corneal Epithelium In Vitro

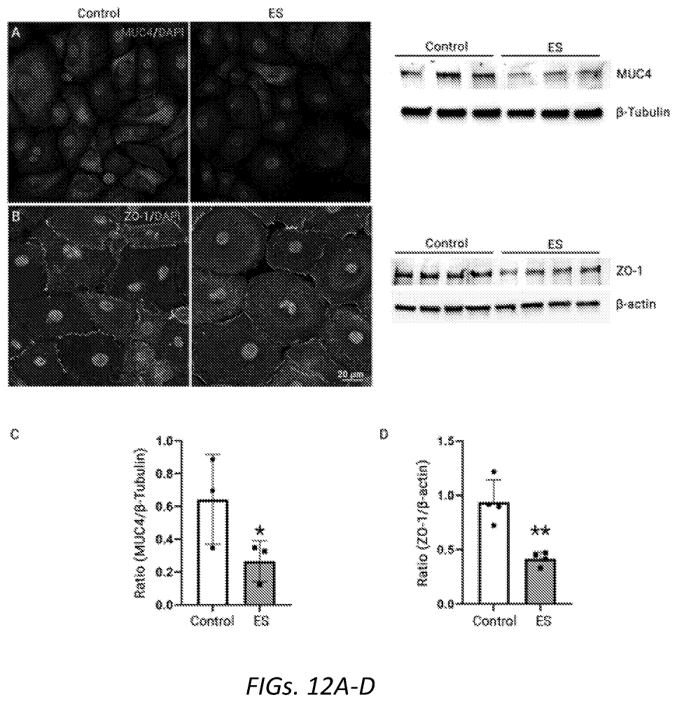

To study the direct actions of ES on the corneal epithelium, we employed ES in PKC cultures. PKC were cultured until the monolayers formed. ES was applied to the monolayer PKC cultures for 30 min each day for 3 days. As PKC monolayer cannot be sustained as a confluent culture for more than a day, longer ES time was employed to compensate for the shortened treatment cycle than used in mice in vivo. Cells were then fixed and analyzed by immunolabeling using primary antibodies against MUC4. MUC4 immunoreactive signal (red) presented as a punctate pattern throughout the cytoplasm in non-ES groups; a reduced MUC4 signal was observed in cells treated with ES (FIG. 12A). As the disruption of tight junctions between corneal epithelium cells is another important hallmark of dry eye, we also investigated the tight junction of PKC using ZO-1 as a marker. ZO-1 immunoreactive signal (green) presented as a serrulate pattern outlining each corneal epithelial cell in the non-ES group; reduced fluorescence signal and blurry lining were observed in cells after ES treatment compared to controls, supporting that ES disrupted tight junctions of PKC (FIG. 12B).

Quantifying MUC4 expression by Western Blots showed a near 60% decrease in MUC4 protein levels in the ES group compared to control (p=0.047, FIG. 12A, right panel). Similarly, the level of ZO-1 protein decreased by 55.55±11.40% in ES groups compared to the control (p=0.0028 FIG. 12B right panel).

We further examined the general transmembrane mucin production or function in cultured PKC using Rose bengal. Rose bengal is an organic anionic dye that stains damaged corneal epithelial cells. The transmembrane mucin expressed on the epithelial cell surface prevents the uptake of rose bengal dye by cells; thus, more rose bengal staining correlates with reduced mucin expression and function. We observed a higher level of rose bengal staining in cells that underwent ES than in controls. We extracted rose bengal dye from the cells and measured the fluorescence intensity at an excitation wavelength of 570 nm using an ELISA reader (BioTek Instruments, Winooski, VT, USA). The fluorescence intensity was significantly higher in extraction from ES-treated cells than in controls (p=0.015). These results supported that ES applied to the corneal epithelial cells negatively impacts the production or function of transmembrane mucin.

### Example 11 TcES Negatively Regulates the Secretion of Secretory Mucin by Inhibiting Intracellular Ca²⁺ ([Ca²⁺]ᵢ) Signaling in Goblet Cells

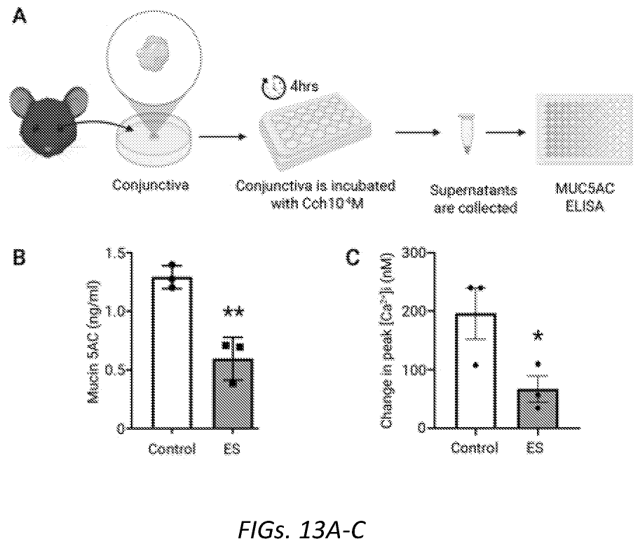

In addition to the decrease in transmembrane mucin caused by ES, we also determined the effect of TcES on the secretory mucin, mainly MUC5AC, from the goblet cells of the conjunctiva in vivo. After 14 days of TcES in vivo, the whole conjunctiva of each animal was dissected and incubated in tear buffer (contains: 1 M NaCl, 0.5 M NaHCO₃, 1M KCl, 1M MgCl₂, 1M NaH2PO₄, 0.5 M HEPES, 1 M CaCl₂)) for 4 h in the presence of a muscarinic agonist carbachol (Cch, 10⁻⁴ M). Cch mimics mucin secretion induced by the efferent parasympathetic nerve that could occur normally. After a 4 h incubation, the supernatant of the conjunctiva was collected, and the amount of MUC5AC was detected using ELISA (FIG. 13A). We noted that conjunctival tissue removed from the TcES eyes showed a significant decrease in the amount of secreted MUC5AC than tissue from the contralateral control eyes (p=0.0043, FIG. 13B).

Because the increase in [Ca²⁺]ᵢ is usually involved in stimulating the secretion of MUC5AC from the conjunctival goblet cells, we investigated goblet cell [Ca²⁺]ᵢ responses after ES. Human primary goblet cells taken from individual donors were seeded in glass-bottomed dishes. Fura2 AM was loaded into the cultured cells 2 h before the [Ca²⁺]ᵢ was measured. One hour after Fura-2 AM addition, ES or control probes without ES were applied to the cultured cells for 1 h. At the end of the treatment, cells were washed and stimulated with Cch (10⁻⁴ M) to mimic the physiological stimulation by the efferent parasympathetic nerves. The [Ca²⁺]ᵢ level over time was recorded, and the change in peak [Ca²⁺]ᵢ was calculated by subtracting the average of the basal value from the peak [Ca²⁺]ᵢ value. In non-ES treated cells, the addition of Cch (10⁻⁴ M) increased the [Ca²⁺]ᵢ to 196.2±43.98 nM above the baseline; while in ES treated cells, the addition of Cch (10⁻⁴ M) increased the [Ca²⁺]ᵢ level to only 67.05±22.24 nM, which is significantly nearly 3× lower than the non-ES treated control (p=0.029, FIG. 13C). These results indicate that TcES negatively regulated the production and release of secretory mucin by decreasing the [Ca²⁺]ᵢ response of conjunctival goblet cells.

### Example 12 TpES does not Induce Corneal Epithelial Damage Nor after the Amount of Corneal MUC4

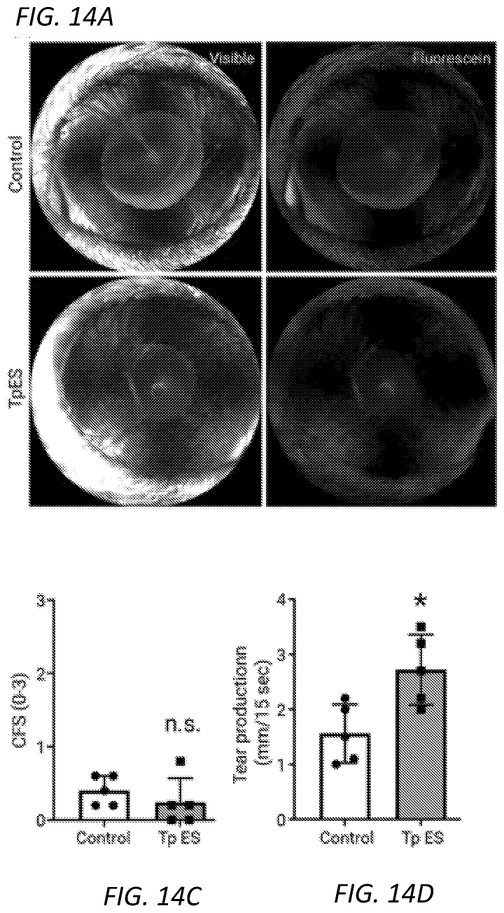

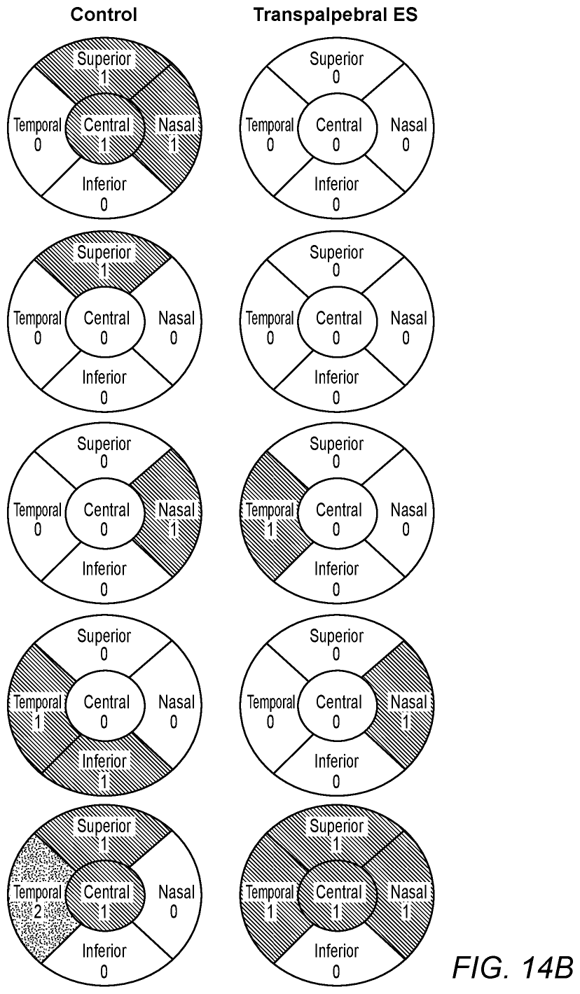

We examined the effect of TpES on the ocular surface. In a similar design, TpES of the same parameter or control probes without ES was applied to the mouse eyelid for 4 min per day for 14 consecutive days. On day 14, corneal fluorescein staining testing was performed. The slit lamp observation showed minimum to no corneal fluorescein staining in both the TpES and control eyes (FIG. 14A). The staining score showed no difference between TpES and control eyes (p=0.38, FIGS. 14B and 14C).

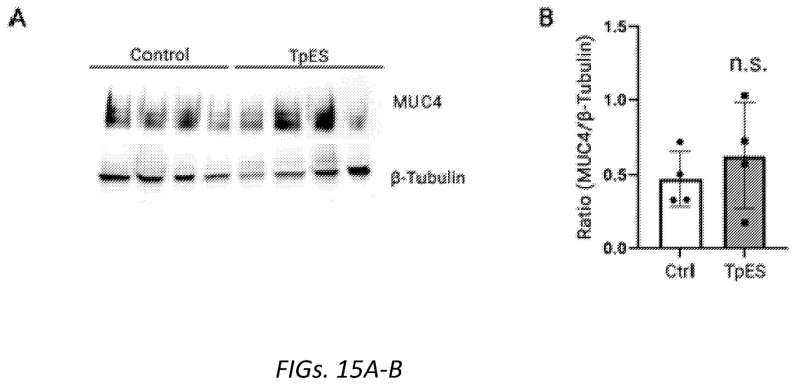

Moreover, phenol-red threat measurement of tear production showed a significant increase in tear production in the TpES eyes compared to the controls (p=0.014, FIG. 14D). In addition, Western Bblot analysis of MUC4 amount was conducted on corneas removed from animals treated with TpES in one eye and no stimulation of the contralateral eye (FIG. 15A). No significant difference was observed in the amount of MUC4 in the TpES corneas compared to corneas from the control eyes (p=0.47, FIG. 15B). In summary, TpES does not cause ocular surface damage nor alter MUC4 expression.

### Example 13 TpES Bypasses the Cornea and Delivers a Higher Electrical Current to the Retina than TcES

To understand why TpES does not cause ocular surface damage and compare the efficacy of electrical current delivery to the retina by TpES and TcES, we assessed the conductive resistances from the stimulation site to the retina.

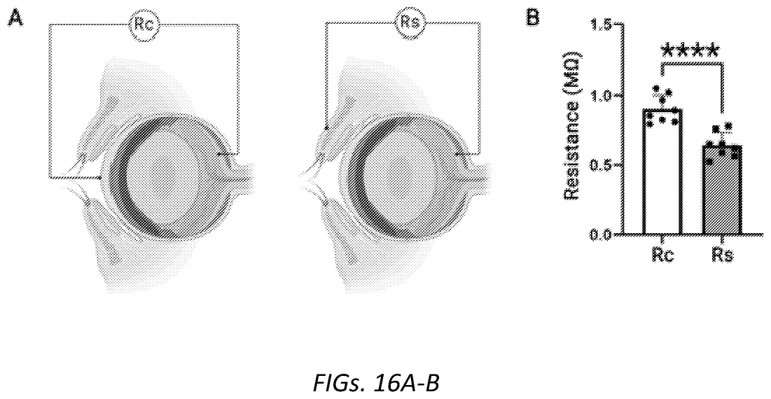

We employed direct current (DC) from the corneal surface to the retina (Rc) or the orbital skin of the upper eyelid to the retina (Rs) of the same eye and measured the total conductive resistance (FIG. 16A). The result shows that Re is 0.90±0.035 MΩ, which is significantly higher than Rs of 0.64±0.032 MΩ (p<0.0001, FIG. 16B), suggesting that TpES is more effective than TcES for delivering electricity to the retina. To understand how much of the electricity delivered via TpES is likely to pass through the cornea, we measured the resistance between the TpES site (orbital skin) and the cornea. We noted that the resistance between the cornea surface and the TpES site is 2.54±0.33 MΩ, ˜4× higher than the resistance measured from the skin to the retina. Given that the conductive resistance from the orbital skin to the cornea is >4 folds larger than that to the retina, we can conclude that no more than 20% electric current goes through the cornea under TpES. Together, our data suggest that while TcES induces ocular surface epithelial damage, TpES may present a better alternative that effectively delivers electricity to the back of the eye without interfering with the mucin homeostasis of the ocular surface.

### Example 14 Ramp Waveform ES is More Effective at Improving Photoreceptor Survival than Rectangular ES in Human Retinal Explant Cultures

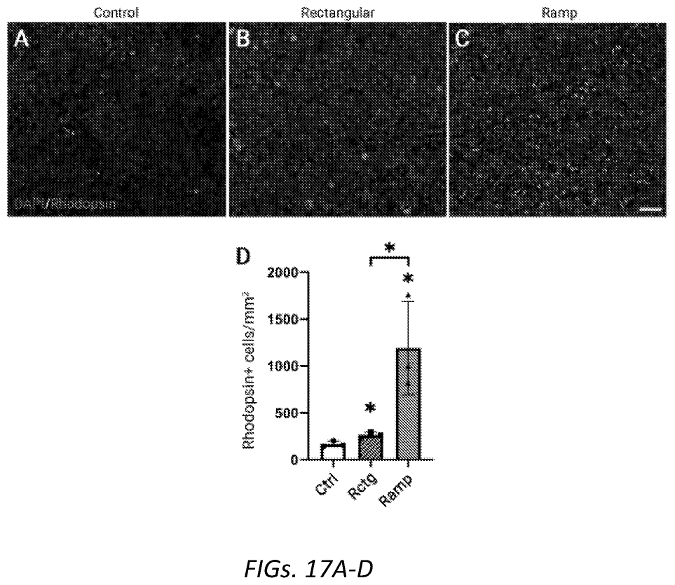

We next compared the neuroprotective potential of different ES waveforms in human post-mortem retinal explant cultures. Our previous studies suggested that the employment of ES at a ramp waveform (20 Hz) or a series of rectangular pulse trains at increasing frequencies from 20-200 Hz improved retinal cell survival in vitro or in vivo. It prompted us to evaluate the effects of these ES conditions in photoreceptor cell survival in culture. Sham-stimulated explants served as the controls. Following 48 hours of incubation after ES, the survival of photoreceptors was assessed using rhodopsin immunostaining. In control cultures, substantial loss of the photoreceptors was observed, with only a few rhodopsin+ cells detectable (FIG. 17A). Rectangular ES significantly improved the number of surviving photoreceptors (FIG. 17B). Remarkably, the stimulation with ramp waveform resulted in dramatically increased photoreceptor survival after culturing (FIG. 17C). Quantitative analysis of the Rhodopsin positive cells in the explants (FIG. 17D) indicated that both rectangular (p<0.05) and ramp waveforms (p<0.05) have a significant increase in the number of rhodopsin positive surviving photoreceptors, while ramp waveform induced over 10 folds increase in the number of surviving photoreceptor cells (p<0.05) compared to the sham control group. These results suggest potent neuroprotective effects by the ramp waveform ES.

### Example 15 Transcorneal ES Improves Retinal Function and Visual Behavior in Rho−/− Mice

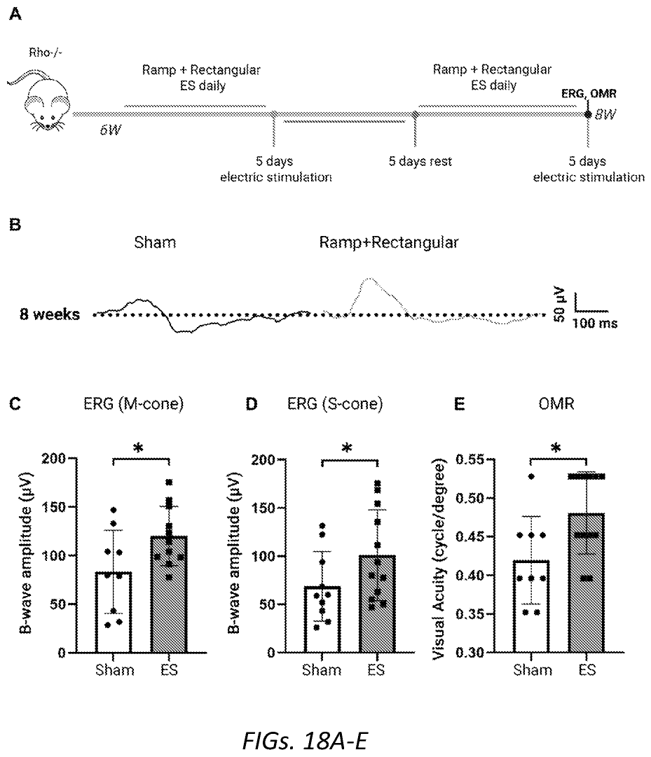

To determine the functional benefits of tES in vivo, we assessed visual perception measured by the optomotor response (OMR)-based assay and quantified photoreceptor survival. Using the previously demonstrated ES paradigm with a current of 100 μA series of rectangular pulse trains with increasing frequencies from 20-200 Hz, we performed tES in Rho−/− mice. Following two 5-day sessions starting at 6 weeks of age, we observed improved photoreceptor survival as previously reported but no significant enhancement of visual acuity in Rho−/− mice. We hypothesized that ES with combined rectangular and ramp waveforms might present a more potent stimulation paradigm in invoking neuroprotective responses and improving retinal and visual function in Rho−/− mice. We employed tES with a combined ramp waveform (100 μA, 20 Hz, 160 seconds) followed by stimulation with the rectangular waveform of 100 μA (40 seconds/cycle, 2-200 Hz, 160 seconds) or sham treatments to one random eye of Rho−/− mice, starting when mice reached 6 weeks old. Following two sessions of five consecutive days tES with a 5-day-rest in between (FIG. 18A), we noted that the eyes received combined tES treatments exhibited significantly improved retinal cone function as measured by ERG than sham-treated mice (FIG. 18B). Significant enhancement of M-cone (FIG. 18C) and S-cone (FIG. 18D) functions was detected two weeks after the first ES treatment (p<0.05). Moreover, we observed significantly (p<0.05) improved visual acuity in ES-treated eyes compared to sham-treated eyes (FIG. 18E). These results indicate functional benefits of tES with combined waveforms of rectangular series and ramp.

### Example 16 tES Attenuates Photoreceptor Loss In Vivo

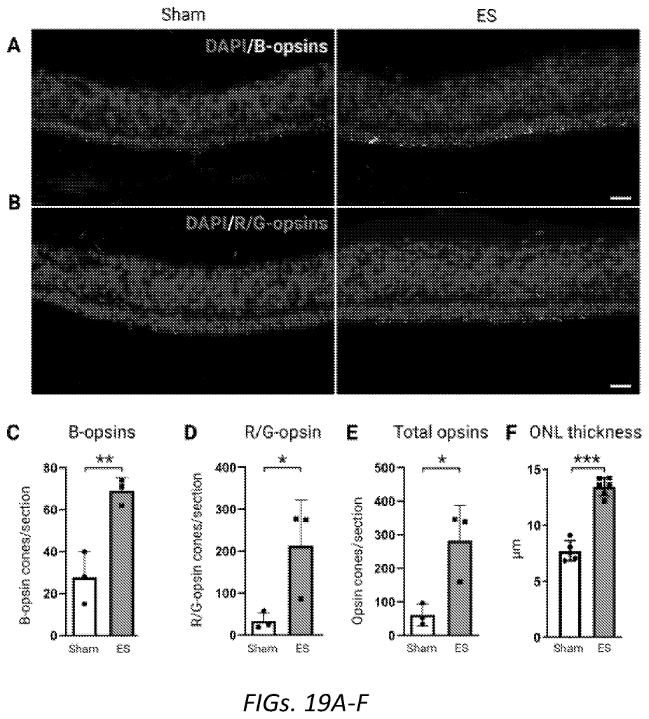

We counted photoreceptor cells, particularly cones, in retinal sections to determine if tES improves retinal and visual functions by preserving photoreceptors because only cones are functional photoreceptors in Rho−/− mice. Immunostaining revealed that tES ameliorated the loss of Blueopsins+ (B-opsins) (FIG. 19A) and Red/Green-opsins+ (R/G-opsins) cells (FIG. 19B). Quantitative analysis demonstrated significantly improved survival of B-opsin-, R/G-opsin- and total Opsin positive cells in the tES group compared to sham-treated contralateral eyes (FIG. 19C-E, p<0.05). Quantifying outer nuclear layer (ONL) thickness also supported significantly increased thicknesses in tES-treated retinas compared to sham-treated eyes, indicating improved survival of all photoreceptor cells after tES treatment (FIG. 19F). Thus, tES ameliorated photoreceptor loss in Rho−/− mice retina.

### Example 17 tES Increased the Density of Bipolar Cell Sprouting and Preserved the Retinal Outer Nuclear Layer

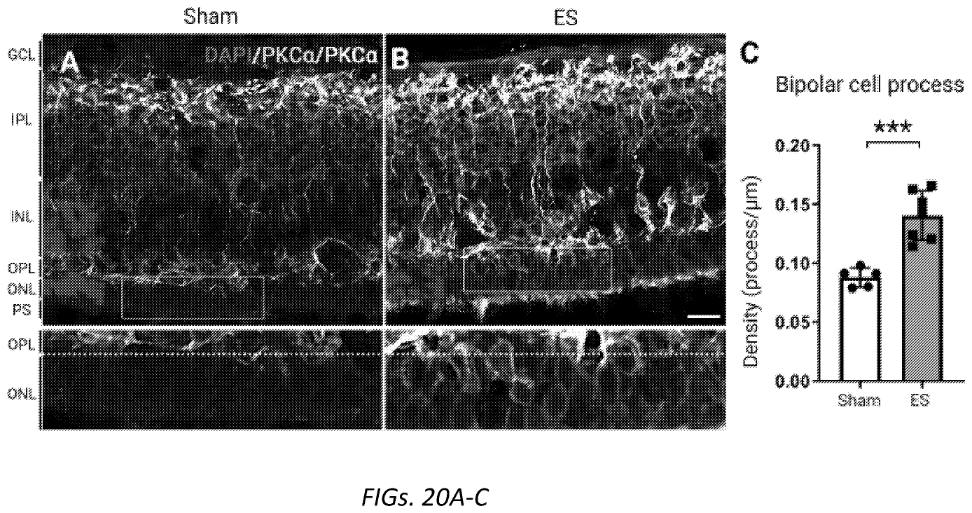

Bipolar cells have been shown to undergo structural changes and neurite sprouting that contribute to retinal function and recovery. To evaluate if tES affected bipolar cell plasticity, we performed immunolabeling for the bipolar cell-specific marker Protein kinase Cα (PKCα). We observed prominent upregulation of PKCα immunolabeling in the tES-treated retina, primarily associated with bipolar cell processes, compared to the sham-treated retina (FIG. 20A-B). We noted an increase in the number of bipolar cell processes that grew into the ONL. Quantitative analysis of the density of the bipolar cell dendrite extending into the ONL the data confirmed that tES significantly increased the density of bipolar cell sprouting into the ONL compared to the sham-treated group (FIG. 20C). Increased retinal plasticity and bipolar cell sprouting in response to the tES may be an additional mechanism contributing to increased photoreceptor survival and function in the Rho−/− mice.

### Example 18 Electric Stimulation Inhibits Microglial Migration, Microvilli Protrusion, and Cytoskeleton Protein Reorganization

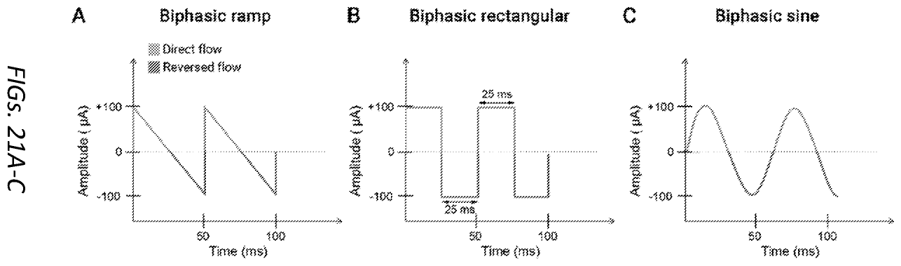

Microglia motility is a strong indicator of its function, particularly in response to pro-inflammatory stimuli (Hikage et al., 2021). To assess the direct impact of the electrical field on microglial function, we investigated the cell motility following ES in cultured BV-2 cells, a murine microglial cell line, using the scratch assay. Our previous studies suggest that while neurons and glial cells respond robustly to electrical microcurrents (100 μA; 20 Hz) (Enayati et al., 2020; Yu et al., 2020), the ramp waveform evokes the most prominent result in glial cells compared to the other waveforms. We compared BV-2's responses to three different ES waveforms ramp (FIG. 21A), rectangular (FIG. 21B), and sine (FIG. 21C). A ˜1 mm scratch was performed in the confluent BV-2 cultures, and cells were subjected to ES (100 μA; 20 Hz) in one of the three waveforms as indicated. Repopulation of the scratch area was observed 24 and 48 h later. Control BV-2 cells cultured in the absence of ES exhibited rapid closure of the scratch distance and disappearance of the surface area within 48 h after scratch (FIG. 21D,H,I). Intriguingly, treatment of BV-2 cells with ES of the ramp waveform, but not rectangular or sine waveforms, resulted in the failure of scratch closure measured at both 24 (p<0.001) and 48 (p<0.001) hours post-ES, when compared with the control group (FIG. 21D-I).

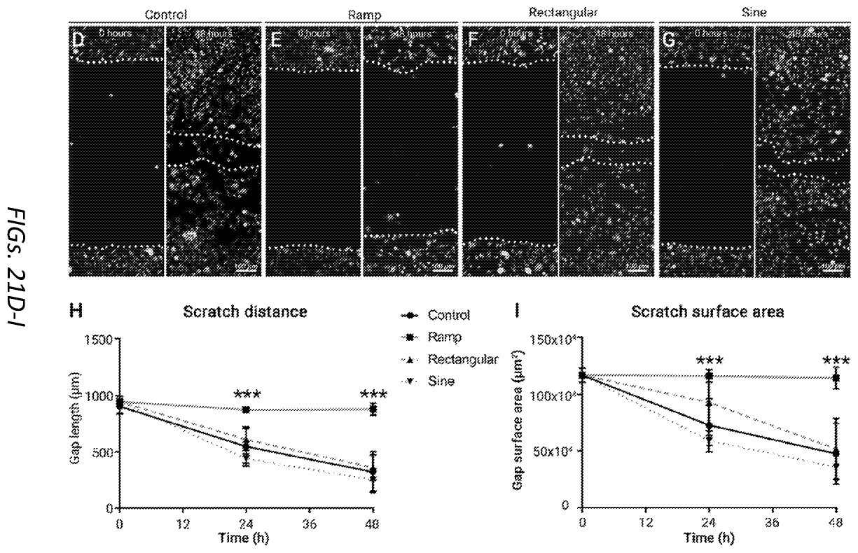

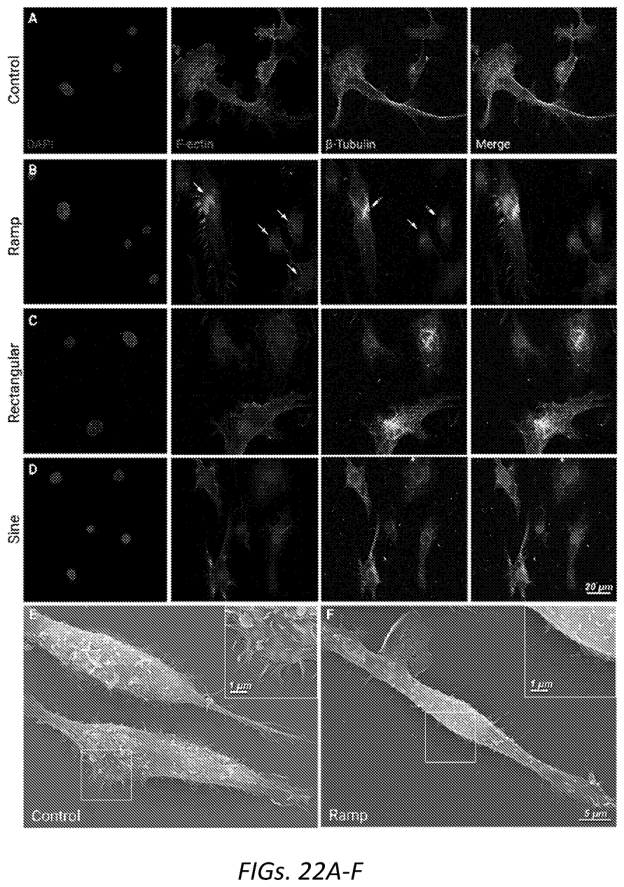

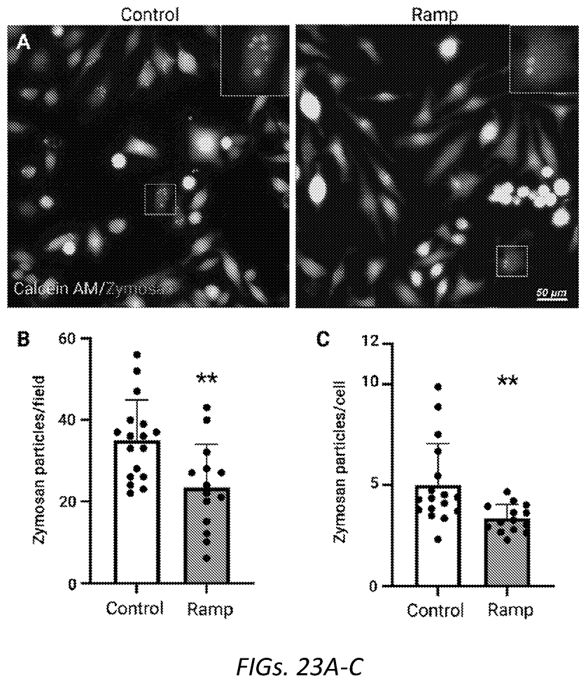

The results indicate a negative impact of ES of a ramp waveform on microglia motility. Dynamic reorganization and rearrangement of actin filaments and microtubules underlie cytoskeletal architectural changes and cell migration (Franco-Bocanegra et al., 2019) Immunocytochemistry of F-actin and β-tubulin in control BV-2 cells revealed cytoskeletal structures extending into the well-defined cellular processes and lamellipodia (FIG. 22A). BV-2 cells received ES of ramp waveform showed diminished presence and redistribution of F-actin (white arrows) and β-tubulin (yellow arrows) with predominant localization to the perinuclear area (FIG. 23B). In contrast, BV-2 cells that received rectangular or sine waveform of ES exhibited similar F-actin and β-tubulin distribution as the control cells, with some changes observed following rectangular ES (FIG. 22B-D). Scanning electron microscopy further demonstrated the formation of lamellipodia and microvilli in control BV-2 cells (FIG. 22E), which were nearly absent in ramp ES treated BV-2 cells (FIG. 22F). Thus, ES with a ramp waveform, but not rectangular and sine, prominently affects F-actin and β-tubulin distribution and organization, reducing the formation of cellular microvilli.

To exclude that ES of ramp waveform induces microglia cytotoxicity, which prevents cell migration and cytoskeleton reorganization, we assessed BV-2 cell survival under various current amplitudes of ramp ES. The cultures of BV-2 cells were treated with ramp microcurrents ranging from 100-1,000 μA. Cell survival was evaluated by measuring Lactate Dehydrogenase (LDH) release 12 h after stimulation. The results indicated no significant increase in cell death at 100 and 250 μA ES compared to control cultures until ES microcurrent reached 500 and 1,000 μA. The results were confirmed using a fluorescent live/death staining kit, in that no significant cell death was observed until the microcurrent reached 500 μA or above. Together, these data suggest that ES with a biphasic ramp waveform, at least when at 100 μA, inhibits the migration and cytoskeleton reorganization of microglia without affecting cell survival.

### Example 19 Electric Stimulation Decreases Microglial Phagocytosis

Microglial phagocytosis is associated with inflammatory responses of microglia and neuron or photoreceptors loss under chronic neurodegenerative conditions, such as retinitis pigmentosa (Zhao et al., 2015). Phagocytosis requires locally coordinated complex cytoskeletal rearrangements (Kuiper et al., 2008); we, therefore, evaluated the phagocytotic activities of BV-2 cells with or without ES treatment by incubating the cultures with fluorescent zymosan particles. Quantification at 24 h post-ES revealed a significant reduction in the total number of fluorescent zymosan particles and zymosan particles per cell (FIG. 23C, p<0.01) in ramp ES-treated BV-2 cells compared to control cultures (FIG. 23). Consistent with our observations of cell motility and cytoskeletal structural changes, rectangular and sine waveforms did not significantly (p>0.05) reduce the number of absorbed fluorescent zymosan particles. However, a tendency of reduction was observed in cultures treated by a rectangular waveform stimulation (p=0.093). Thus, ES-treatment with ramp waveform directly inhibits the phagocytotic activities of microglia. From hereon, only ES with a ramp waveform was used and analyzed.

### Example 20 Electric Stimulation Attenuates LPS-Induced Production of Pro-Inflammatory Cytokines by Activated Microglia

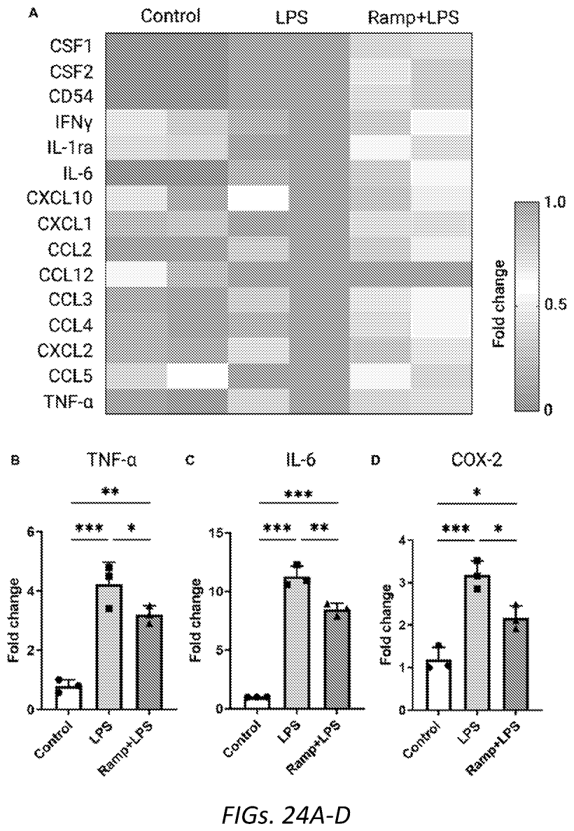

Microglia motility and phagocytotic capacity are important aspects of their pro-inflammatory activation. Microglial activation produces pro-inflammatory cytokines such as IL-6 and TNF-α that can be neurotoxic and contribute to the development and progression of neurodegenerative conditions in the brain and retina (Rashid et al., 2019). We next studied the effects of ES on LPS-induced pro-inflammatory cytokine release in activated microglia using a proteome profiler mouse cytokine array. The BV-2 cells were stimulated with ramp ES, followed by incubation with LPS (1 μg/ml), a widely recognized potent activator of microglia (Lively and Schlichter, 2018). As expected, LPS induced the expression of pro-inflammatory cytokines, including Macrophage colony-stimulating factor 1 (CSF1), Macrophage colony-stimulating factor 2 (CSF2), IL-6, IFNγ, TNFα, and C—C Motif Chemokine ligands (CCL) 2, 3, four and 5. ES treatment attenuated the production of all proinflammatory cytokines induced by LPS (FIG. 24A). ES attenuation of LPS-induced microglial activation was further validated by qPCR of TNFα (FIG. 24B; p<0.05), IL-6 (FIG. 24C; p<0.01), and COX-2 (FIG. 24D, p<0.05). Together, these results indicate robust negative regulatory effects of ES on microglia activation and pro-inflammatory cytokine production.

### Example 21 Electric Stimulation Attenuates LPS-Induced Transcriptome Profile Changes in Microglial Cells

To comprehensively study the effects of ES in microglia, we performed RNA sequencing. The RNA specimens were extracted from BV-2 cultures treated with ramp ES, followed by a challenge with LPS. Cultures challenged by LPS alone were served as positive controls, and those treated by PBS were used as negative controls.

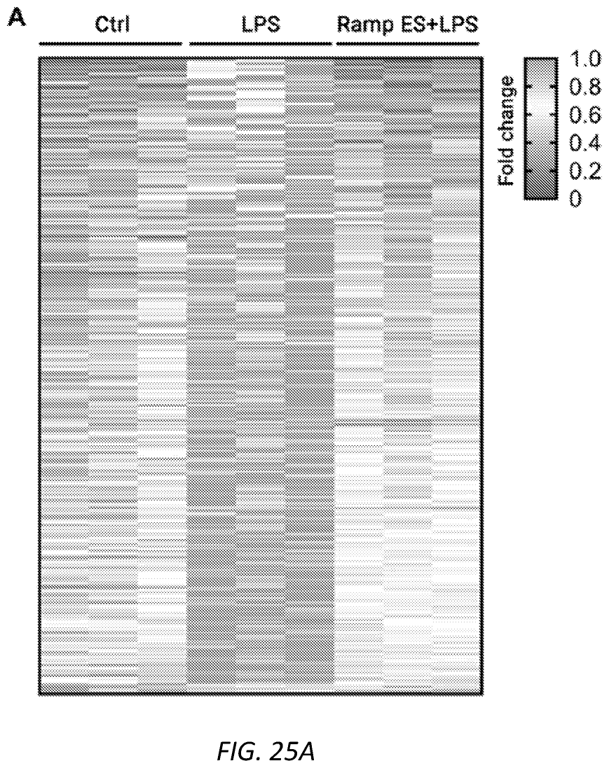

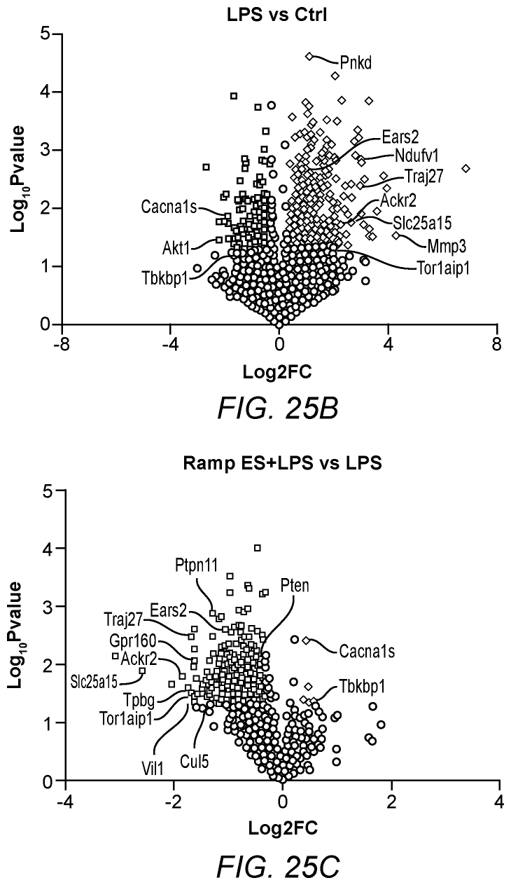

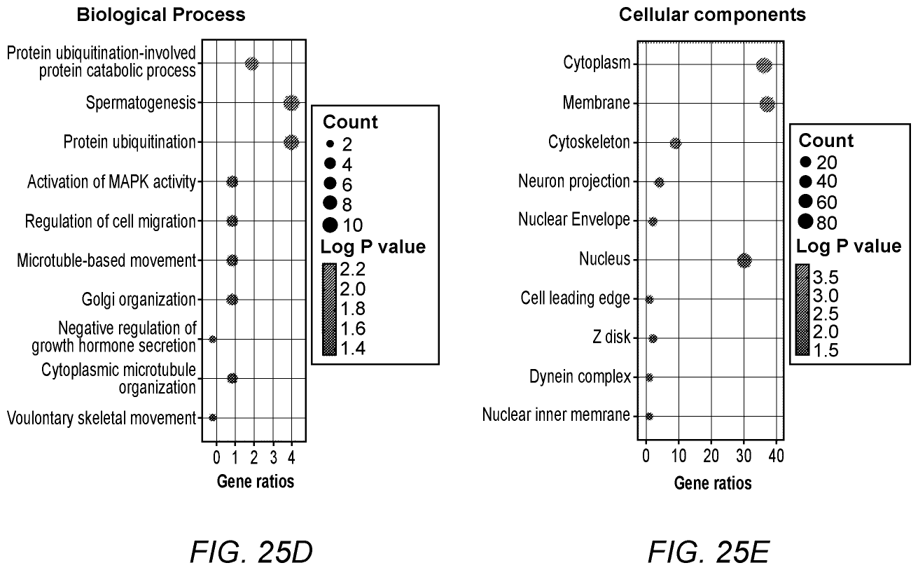

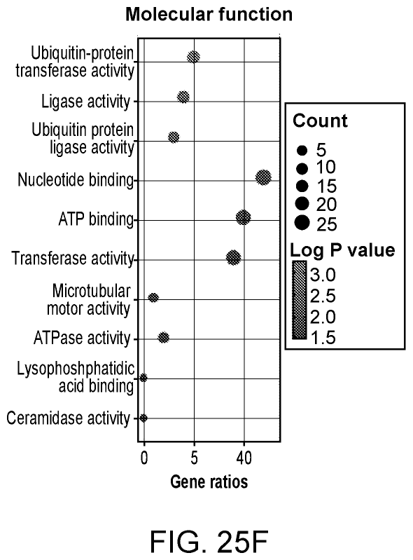

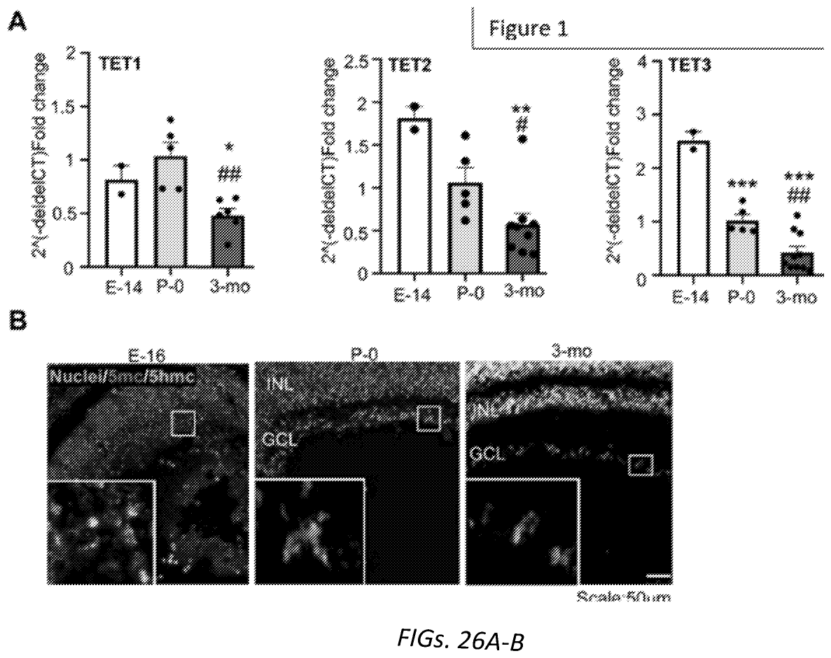

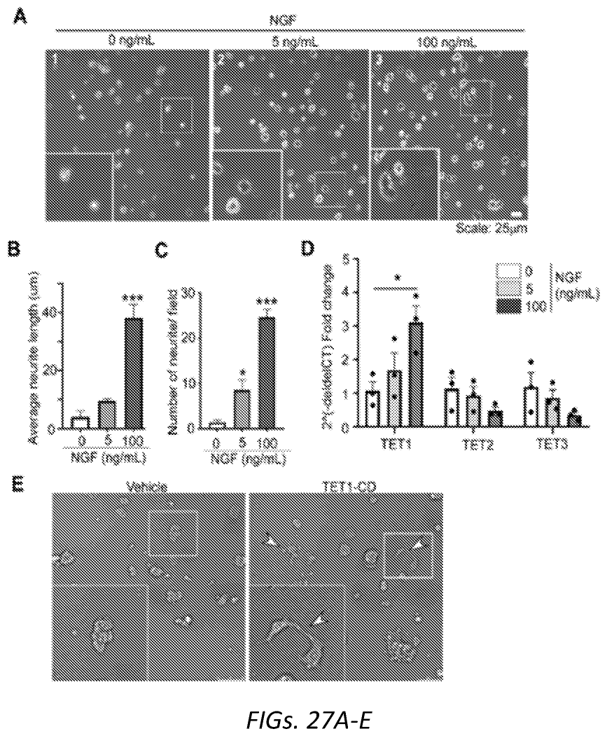

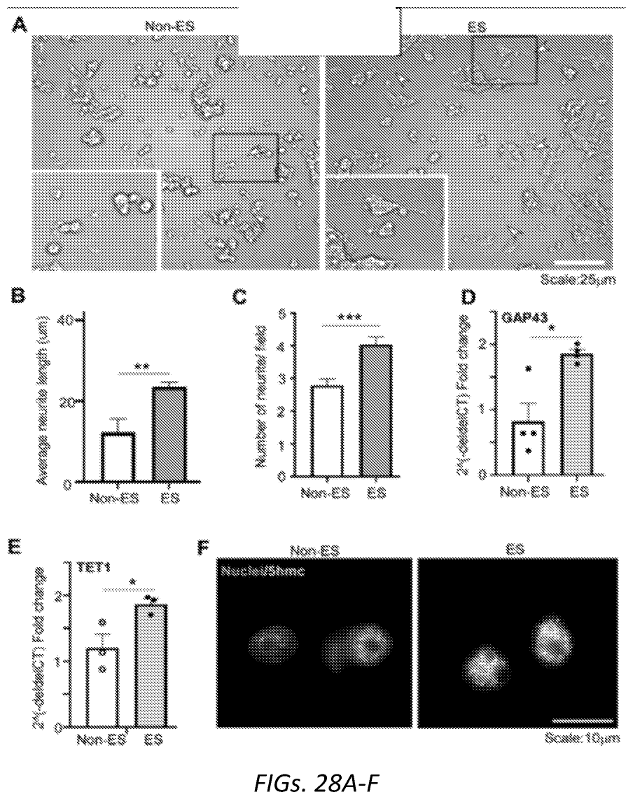

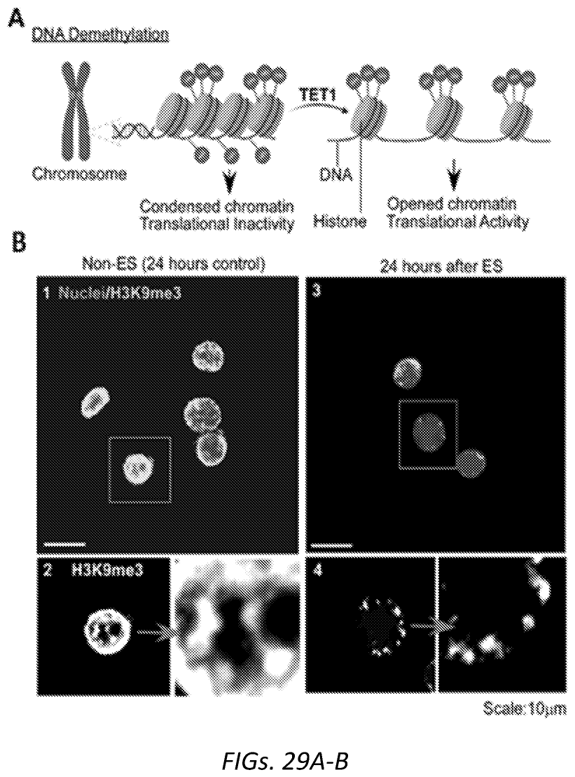

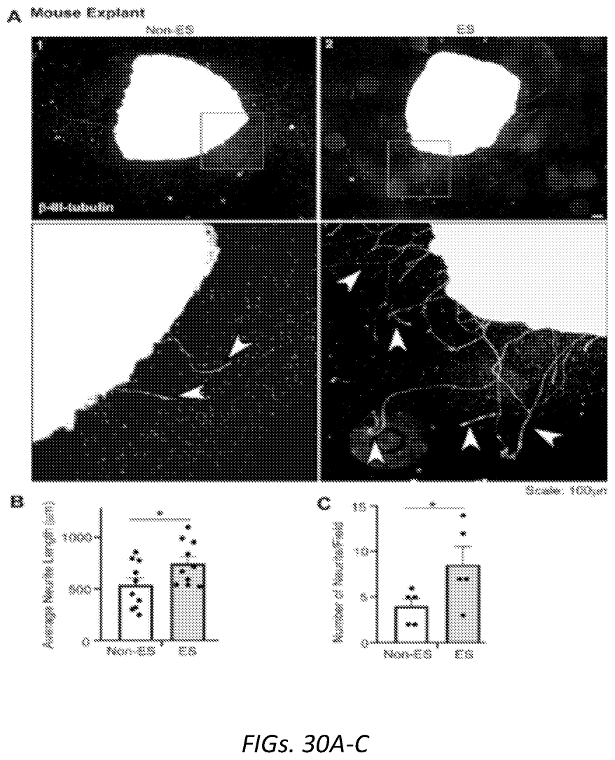

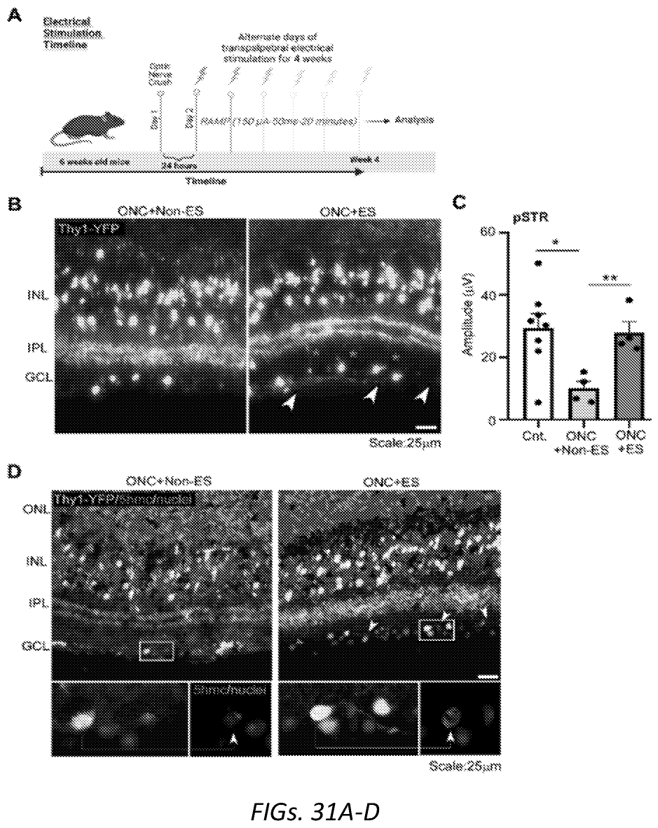

Following RNA-seq data normalization, we achieved a clear separation between the populations of the experimental groups in the principal component (PC) analysis. Using the cutoff of p-value <0.05 and fold changes >1.2, we detected 923 differentially expressed genes (DEGs)—686 upregulated and 237 downregulated genes—induced by LPS compared to PBS-treated controls. LPS treatment induced a pro-inflammatory DEG profile, showing increases in Matrix metalloproteinase (Mmp3), energy metabolism-related genes (Slc25a15, Ears2, Ndufv1), inflammation-related genes (IL-6, TNFα, Traj27, Ackr2), and others (FIG. 25B). The microglia cell ATPase activity (DYNC2H1, DYNC1H1, DNAH12, MACF1, DDX3X, ABCB1A, ABCF3, MDN1); ATP binding (CAMK2D, DDX3X, KIF11, TTBK2, GK5, SLK, ACVR1C, BTAF1, ATP2B4, and others) as well as NADP binding (DHFR, ME1, FMO2, PGD) were upregulated in LPS treated group relative to control. Furthermore, microglia motility-related (DYNC2H1, DYNC1H1, DNAH12, STARD9. KIF11) genes were upregulated (Supplementary file DEG_LPSvsCtrl). In contrast, ES pretreatment to LPS-challenged (Supplementary file DEG_LPSvsCtrl.xls) BV-2 cultures significantly suppressed 294 out of 923 LPS-induced DEGs. These include the expression of the above-mentioned energy (Supplementary file DEG_ES_LPSvsLPS.xlsx) metabolism and motility-related genes that were upregulated by LPS. Inflammation-related genes such as (Traj27, Ackr2) were also downregulated while inducing only a few gene upregulations (FIG. 25C). Only six genes were significantly upregulated in the ES pretreatment to LPS-challenged; they included L-type voltage-dependent calcium channel alpha 1S (Cacnals), TBK Binding Protein 1 (Tbkbp1), Zfp771), and three unknown transcripts Gm38134, Gm13432, Gm28959 (FIG. 5C; Supplementary Tables 51-3). The RNA-seq data were further validated by qPCR of selected genes, including Tbkbp1 (p=0.047; Log 2 FC=0.481; Supplementary Figure S4A) and Atp2b4 (p=0.022; Log 2 FC=−0.997). The gene ontology (GO) analysis of the ES-downregulated DEGs compared to the LPS-challenged group revealed decreases in cell migration, microtubule-based movement (FIG. 25D), cytoskeleton (FIG. 25E), and microtubule motor activity-related genes (FIG. 25E); nearly all of this genes are significantly upregulated in BV-2 cells challenge with LPS when compared with control. These results align with the above observations that ES reduced microglial motility. Furthermore, ES also decreased DEGs associated with ATPase activity (FIG. 25E), ATP binding (FIG. 25E), and NADP binding—genes underlying the control of cellular metabolism and oxygen consumption. The number of metal ion binding genes (KDM5A, NRP2, GALNT15, HHIP, FCNA, ENDOV, MET, TRIM24, PAPOLA, ZBTB39, DDX59, MBNL1) were also significantly downregulated by ES pretreatment while upregulated in LPS challenged group relative to control. Together, these data further support the prominent negative effects of ES on LPS-induced microglia motility, metabolism changes, and neuroinflammatory responses.

### Example 22 Transpalpebral Electrical Stimulation Enhances Corneal Nerve Regeneration

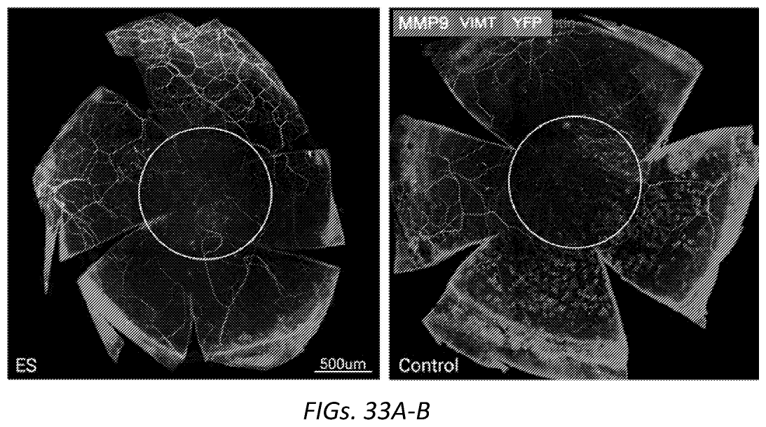

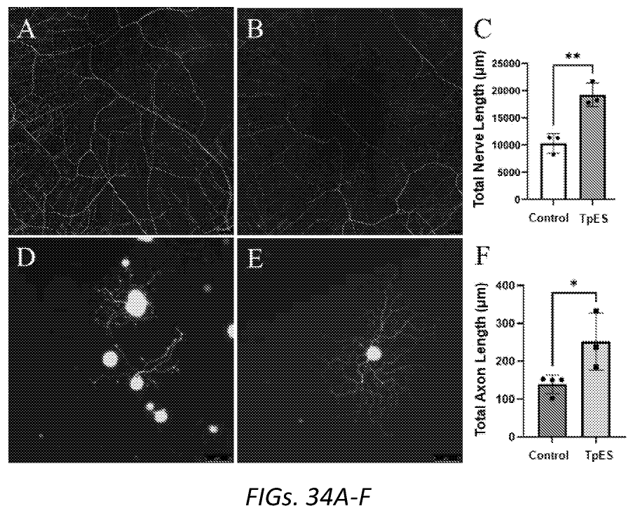

Eight to twelve week-old female B6.Cg-Tg(Thy1-YFP)16Jrs/J mice were used to examine corneal nerve regeneration. Thy-1 is a cell surface glycoprotein expressed on neurons, allowing the visualization of the corneal nerves. The epithelium of the cornea and the superficial stroma of an eye was removed and confirmed by corneal fluorescent staining. TpES was applied with ramp waveform at 20 Hz, 300 uA to the injured eye for 4 min a day, for 14 days, with the age/sex-matched non-ES mice as control. NeuroJ was used for image analysis. p<0.05 was considered significant. The corneas were collected on day 14, and the corneal wholemount was conducted. On day 14, the eyes that underwent TpES showed significant renervation covering the center of the cornea, while minimum to no nerve was observed in the central cornea in control (FIGS. 33A-B). The total nerve length of the entire cornea was significantly higher in TpES cornea compared to the control (p<0.05). Moreover, the markers for corneal myofibroblast activation and extracellular matrix remodeling, Vimentin, and Matrix metalloproteinase (MMP)-9 were significantly reduced in TpES corneas compared to control. This indicates an anti-fibrosis effect of TpES in corneal wound and scar formation. Additionally, when TpES was applied for 21 days with ramp waveform at 20 Hz, 300 uA, the central cornea had longer nerve lengths compared to control (FIG. 34C). Neurons were isolated from TGs from the same Thy-1-YFP mice for 24 hours. The total length of the axons was significantly greater in ES-treated neurons than control (FIG. 34F) The TGs of the TpES side were also collected, and the neurons were cultured onto glass-bottomed dishes for imaging. 24 h into the culture, axons were observed.

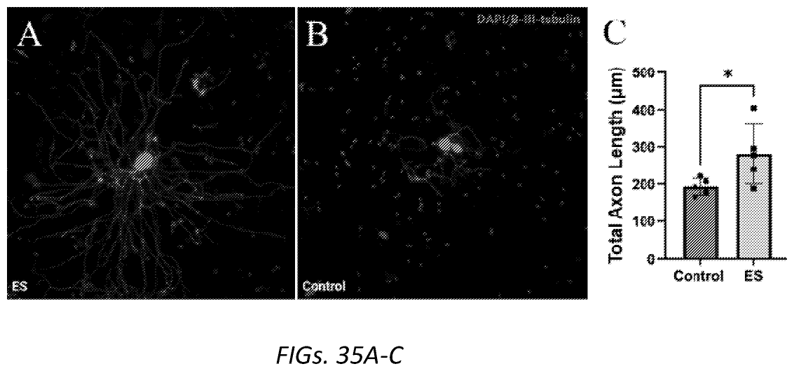

Significant growth of axons was observed in TGs underwent TpES compared to the control (FIG. 35A, B). TGs of non-ESed animals were cultured and ES was applied in vitro, for 30 min, with ramp waveform at 20 Hz, 100 uA. ES significantly enhanced the growth of axons compared to non-ES TGs. (FIG. 35C)

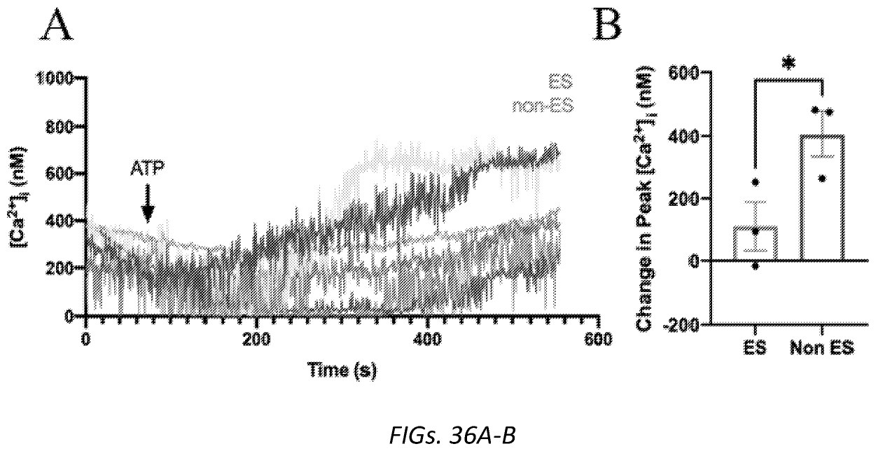

Intracellular [Ca²⁺]([Ca²⁺]ᵢ) level represents neuron pain-sensing activity of cultured TG neurons in real-time with the application of ES (ramp, 100 uA, 20 Hz). The TG cells were stimulated with adenosine triphosphate (ATP) and the [Ca²⁺]ᵢ was measured before and after the addition of ATP (10⁻⁷M; FIG. 36A). The ATP-induced change of [Ca²⁺]ᵢ was significantly lower with ES treatment than in non-ES treated TG cells, indicating a potential corneal pain control effect of ES (FIG. 36B).

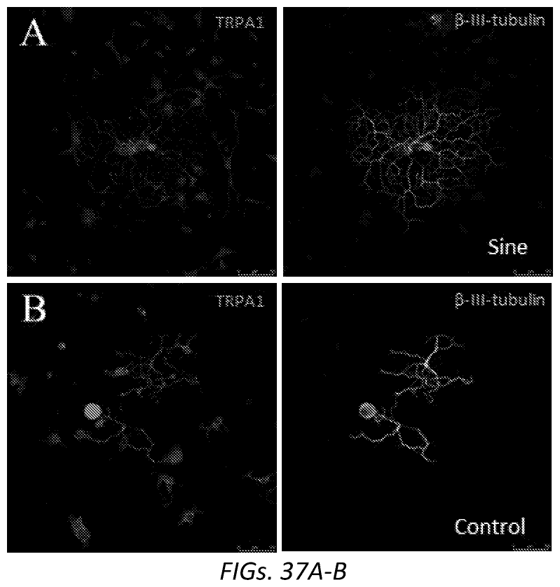

Additionally, a sine waveform (20 Hz, 100 uA) was applied to cultured TGs in vitro, for 30 min. Similar results were obtained with sine waveform ES significantly enhancing the growth of axons compared to non-ES TGs, while also down-regulating the expression of nonreceptor TRPA1. This result indicates sine waveform also has the potential to enhance corneal nerve regeneration and reduce corneal pain (FIG. 37 A, B).

### Example 23 Transpalpebral Electrical Stimulation Reduces Injury and Preserves Function in Models of Ischemia/Reperfusion

Ischemia/reperfusion (I/R) injury was induced in adult C57BL/6J mice by unilateral acute elevation of intraocular pressure to 75 mmHg through anterior chamber cannulation with normal saline for 1 hour. Biphasic ES (n=11) was given transpalpebrally on 4 equal distance spots around the affected mouse eye for 1 min/spot. A control group of mice (n=9) with I/R injury received a sham procedure. ES was conducted every other day starting 24 hours after I/R for a period of 4 weeks. Retinal and visual functions were evaluated by electroretinogram (ERG) and optomotor response (OMR) weekly. Mice were sacrificed 4 weeks after I/R injury for immunohistochemistry. Statistical significance was calculated using a Student's t-test or one-way ANOVA, and a value of p<0.05 was considered statistically significant.

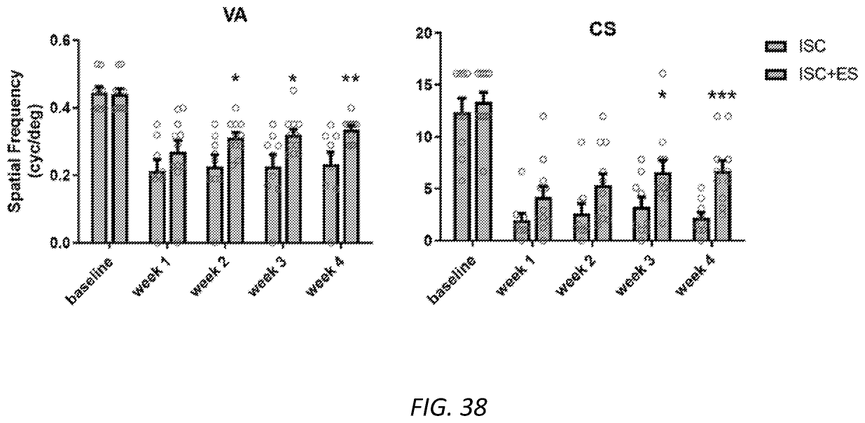

The induction of I/R injury caused a drastic decrease in both the retinal and visual functions due to the ischemic damage to the retina. ES treatment significantly improved spatial-visual functions, including visual acuity (VA) and contrast sensitivity (CS), as early as 2 weeks after I/R injury compared to sham controls (FIG. 38). When compared to baseline, the reduction of both VA and CS were dramatic in the Sham-treated mice from week 1 through week 4 (P<0.0001; one-way ANOVA). However, in the group that received ES treatment, both VA and CS slowly improved over time. Notably, the VA in the ES-treated group started with a P value <0.001 in week 1, P<0.05 in weeks 2 and 3, and become not statistically significant in week 4 when compared to the baseline (one-way ANOVA). These results indicate that applying transpalpebral ES improves VA over time.

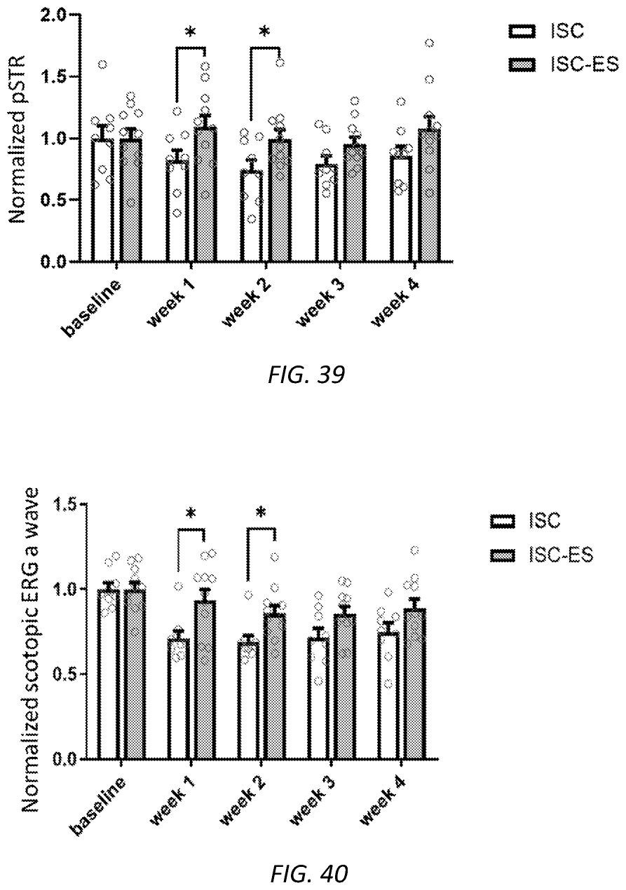

Concurrently, ES retained retinal functions of positive scotopic threshold response (pSTR) (FIG. 39) and promoted rod photoreceptor function (FIG. 40) significantly in I/R mice over time compared to the sham treatment group. The pSTR is primarily associated with the function of retinal ganglion cells (RGC), which are involved in transmitting visual information from the retina to the brain. After inducing I/R injury, the pSTR function of sham-treated mice dropped about 25% in week 2 after normalizing to baseline. RGC function was maintained in the ES-treated group but not in the sham-treated group, as the pSTR for the ES-treated group was maintained similarly to the normalized baseline level over time (p<0.05; Student's t-test as compared to sham-treated group at same time point; FIG. 39). Further, ES also maintained rod photoreceptors functioning in I/R mice over time, as demonstrated in the quantification of scotopic electroretinogram (ERG) a wave analysis respectively. Significant improvements in rod photoreceptor function were found in the ES-treated group in the first 2 weeks compared to the sham-treated mice (p<0.05; Student's t-test; FIG. 40).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### REFERENCES

1. Akpek E K, Amescua G, Farid M, Garcia-Ferrer F J, Lin A, Rhee M K, Varu D M, Musch D C, Dunn S P, Mah F S et al: Dry Eye Syndrome Preferred Practice Pattern®. Ophthalmology 2019, 126(1):P286-P334.
2. Argueso P, Tisdale A, Spurr-Michaud S, Sumiyoshi M, Gipson I K: Mucin characteristics of human corneal-limbal epithelial cells that exclude the rose bengal anionic dye. Invest Ophthalmol Vis Sci 2006, 47(1):113-119.
3. Aum D J, Tierney T S: Deep brain stimulation: foundations and future trends. Front Biosci (Landmark Ed) 2018, 23(1):162-182.
4. Berglund K, Midorikawa M, Tachibana M. Increase in the pool size of releasable synaptic vesicles by the activation of protein kinase C in goldfish retinal bipolar cells. J Neurosci 2002; 22:4776-4785.
5. Bittner A K, Seger K, Salveson R, et al. Randomized controlled trial of electro-stimulation therapies to modulate retinal blood flow and visual function in retinitis pigmentosa. Acta Ophthalmol 2018; 96:e366-e376.
6. Boutouja F, Stiehm C M, Platta H W: mTOR: A Cellular Regulator Interface in Health and Disease. Cells 2019, 8(1).
7. Cao W, Wen R, Li F, Cheng T, Steinberg RH. Induction of basic fibroblast growth factor mRNA by basic fibroblast growth factor in Muller cells. Invest Ophthalmol Vis Sci 1997; 38:1358-1366.
8. Cehajic-Kapetanovic J, Xue K, Martinez-Fernandez de la Camara C, et al. Initial results from a first-in-human gene therapy trial on X-linked retinitis pigmentosa caused by mutations in RPGR. Nature Medicine 2020; 26:354-359.
9. Chaikin L, Kashiwa K, Bennet M, Papastergiou G, Gregory W.

Microcurrent stimulation in the treatment of dry and wet macular degeneration. Clin Ophthalmol 2015; 9:2345-2353.
10. Chow A Y, Chow V Y, Packo K H, Pollack J S, Peyman G A, Schuchard R. The artificial silicon retina microchip for the treatment of vision loss from retinitis pigmentosa. Arch Ophthalmol 2004; 122:460-469.
11. Chu E C, Tarnawski A S: PTEN regulatory functions in tumor suppression and cell biology. Med Sci Monit 2004, 10(10):RA235-241.
12. Corfield A P: Mucins: a biologically relevant glycan barrier in mucosal protection. Biochim Biophys Acta 2015, 1850(1):236-252.
13. Craig J P, Nelson J D, Azar D T, Belmonte C, Bron A J, Chauhan S K, de Paiva C S, Gomes J A P, Hammitt K M, Jones L et al: TFOS DEWS II Report Executive Summary. Ocul Surf 2017, 15(4):802-812.
14. Craig J P, Nichols K K, Akpek E K, Caffery B, Dua H S, Joo C K, Liu Z, Nelson J D, Nichols J J, Tsubota K et al: TFOS DEWS II Definition and Classification Report. Ocul Surf 2017, 15(3):276-283.
15. Daiger S P, Sullivan L S, Bowne S J. Genes and mutations causing retinitis pigmentosa. Clin Genet 2013; 84:132-141.
16. Dartt D A: Regulation of mucin and fluid secretion by conjunctival epithelial cells. Prog Retin Eye Res 2002, 21(6):555-576.
17. Enayati S, Chang K, Achour H, Cho K S, Xu F, Guo S, K Z E, Xie J, Zhao E, Turunen T et al: Electrical Stimulation Induces Retinal Muller Cell Proliferation and Their Progenitor Cell Potential. Cells 2020, 9(3).
18. Enayati S, Chang K, Achour H, et al. Electrical Stimulation Induces Retinal Muller Cell Proliferation and Their Progenitor Cell Potential. Cells 2020; 9.
19. Fish R M, Geddes L A: Conduction of electrical current to and through the human body: a review. Eplasty 2009, 9:e44.
20. Fujikado T, Morimoto T, Matsushita K, Shimojo H, Okawa Y, Tano Y.

Effect of transcorneal electrical stimulation in patients with nonarteritic ischemic optic neuropathy or traumatic optic neuropathy. Jpn J Ophthalmol 2006; 50:266-273.
21. Gipson I K: Goblet cells of the conjunctiva: A review of recent findings. Prog Retin Eye Res 2016, 54:49-63.
22. Greferath U, Grunert U, Wassle H. Rod bipolar cells in the mammalian retina show protein kinase C-like immunoreactivity. J Comp Neurol 1990; 301:433-442.
23. Guzman-Aranguez A, Argueso P: Structure and biological roles of mucin-type O-glycans at the ocular surface. Ocul Surf 2010, 8(1):8-17.
24. Hamel C. Retinitis pigmentosa. Orphanet Journal of Rare Diseases 2006:1:40.
25. Haq W, Dietter J, Zrenner E. Electrical activation of degenerated photoreceptors in blind mouse retina elicited network-mediated responses in different types of ganglion cells. Sci Rep 2018; 8:16998.
26. Hartong D T, Berson E L, Dryja T P. Retinitis pigmentosa. Lancet 2006:368:1795-1809.
27. Hiemer B, Krogull M, Bender T, Ziebart J, Krueger S, Bader R, Jonitz-Heincke A: Effect of electric stimulation on human chondrocytes and mesenchymal stem cells under normoxia and hypoxia. Mol Med Rep 2018, 18(2):2133-2141.
28. Hodges R R, Dartt D A: Tear film mucins: front line defenders of the ocular surface; comparison with airway and gastrointestinal tract mucins. Exp Eye Res 2013, 117:62-78.
29. Huang S, Tang N, Zhao H, Tang C L: Effect of electrical stimulation combined with diet therapy on insulin resistance via mTOR signaling. Mol Med Rep 2019, 20(6):5152-5162.
30. Humphries M M, Rancourt D, Farrar G J, et al. Retinopathy induced in mice by targeted disruption of the rhodopsin gene. Nat Genet 1997; 15:216-219.
31. Jin J, Sklar G E, Min Sen Oh V, Chuen Li S. Factors affecting therapeutic compliance: A review from the patient's perspective. Ther Clin Risk Manag 2008; 4:269-286.
32. Jones B W, Pfeiffer R L, Ferrell W D, Watt C B, Marmor M, Marc R E.

Retinal remodeling in human retinitis pigmentosa. Exp Eye Res 2016; 150:149-165.
33. Karschin A, Wassle H. Voltage- and transmitter-gated currents in isolated rod bipolar cells of rat retina. J Neurophysiol 1990; 63:860-876.
34. Kautto L, Nguyen-Khuong T, Everest-Dass A, Leong A, Zhao Z, Willcox M D P, Packer N H, Peterson R: Glycan involvement in the adhesion of Pseudomonas aeruginosa to tears. Exp Eye Res 2016, 145:278-288.
35. Kolb H, Zhang L, Dekorver L. Differential staining of neurons in the human retina with antibodies to protein kinase C isozymes. Vis Neurosci 1993:10:341-351.
36. Kurimoto T, Oono S, Oku H, et al. Transcorneal electrical stimulation increases chorioretinal blood flow in normal human subjects. Clin Ophthalmol 2010:4:1441-1446.
37. Lee S, Lee C, Kim E, Ko S A, Kim S N, Choy Y B, Im C H: In-vivo estimation of tissue electrical conductivities of a rabbit eye for precise simulation of electric field distributions during ocular iontophoresis. Int J Numer Method Biomed Eng 2022, 38(1):e3540.
38. Lee S, Park J, Kwon J, Kim D H, Im C H: Multi-channel transorbital electrical stimulation for effective stimulation of posterior retina. Sci Rep 2021, 11(1):9745.
39. Lennikov A, Yang M. Chang K, et al. Direct modulation of microglial function by electrical field. Frontiers in Cell and Developmental Biology 2022; 10.
40. Lennikov A, Yang M. Chang K, Pan L, Saddala M S, Lee C, Ashok A, Cho K-S, Utheim T P, Chen D F: Direct modulation of microglial function by electrical field. Frontiers in Cell and Developmental Biology 2022, 10.
41. Lewis G P, Linberg K A, Fisher S K. Neurite outgrowth from bipolar and horizontal cells after experimental retinal detachment. Invest Ophthalmol Vis Sci 1998:39:424-434.
42. Machiele R, Lopez M J, Czyz C N: Anatomy, Head and Neck, Eye Lacrimal Gland. In: StatPearls. edn. Treasure Island (F L); 2022.
43. Moisset X, Lefaucheur J P: Non pharmacological treatment for neuropathic pain: Invasive and non-invasive cortical stimulation. Rev Neurol (Paris) 2019, 175(1-2):51-58.
44. Morimoto T, Fujikado T, Choi J S, et al. Transcorneal electrical stimulation promotes the survival of photoreceptors and preserves retinal function in royal college of surgeons rats. Invest Ophthalmol Vis Sci 2007; 48:4725-4732.
45. Morimoto T, Miyoshi T, Matsuda S, Tano Y, Fujikado T, Fukuda Y. Transcorneal electrical stimulation rescues axotomized retinal ganglion cells by activating endogenous retinal IGF-1 system. Invest Ophthalmol Vis Sci 2005:46:2147-2155.
46. Morimoto T, Miyoshi T, Sawai H, Fujikado T. Optimal parameters of transcorneal electrical stimulation (TES) to be neuroprotective of axotomized RGCs in adult rats. Exp Eye Res 2010; 90:285-291.
47. Navntoft C A, Marozeau J, Barkat T R. Ramped pulse shapes are more efficient for cochlear implant stimulation in an animal model. Sci Rep 2020; 10:3288.
48. Naycheva L, Schatz A, Willmann G, Bartz-Schmidt K U, Zrenner E, Rock T, Gekeler F: Transcorneal electrical stimulation in patients with retinal artery occlusion: a prospective, randomized, sham-controlled pilot study. Ophthalmol Ther 2013, 2(1):25-39.
49. Ota Y, Ozeki N, Yuki K, Shiba D, Kimura I, Tsunoda K, Shinoda K, Ohde H, Tsubota K: The Efficacy of Transcorneal Electrical Stimulation for the Treatment of Primary Open-angle Glaucoma: A Pilot Study. Keio J Med 2018, 67(3):45-53.
50. Ozaki E, Delaney C, Campbell M, Doyle S L. Minocycline suppresses disease-associated microglia (DAM) in a model of photoreceptor cell degeneration. Experimental Eye Research 2022; 217:108953.
51. Percie du Sert N, Hurst V, Ahluwalia A, Alam S, Avey M T, Baker M, Browne W J, Clark A, Cuthill I C, Dimagl U et al: The ARRIVE guidelines 2.0: Updated guidelines for reporting animal research. PLoS Biol 2020, 18(7):e3000410.
52. Reis C, Arruda B S, Pogosyan A, Brown P, Cagnan H: Essential tremor amplitude modulation by median nerve stimulation. Sci Rep 2021, 11(1):17720.
53. Saxton R A, Sabatini D M: mTOR Signaling in Growth, Metabolism, and Disease. Cell 2017, 168(6):960-976.
54. Scangos K W, Khambhati A N, Daly P M, Makhoul G S, Sugrue L P, Zamanian H, Liu T X, Rao V R, Sellers K K, Dawes H E et al: Closed-loop neuromodulation in an individual with treatment-resistant depression. Nat Med 2021, 27(10):1696-1700.
55. Scangos K W, Makhoul G S, Sugrue L P, Chang E F, Krystal A D: State-dependent responses to intracranial brain stimulation in a patient with depression. Nat Med 2021, 27(2):229-231.
56. Schatz A, Pach J, Gosheva M, Naycheva L, Willmann G, Wilhelm B, Peters T, Bartz-Schmidt K U, Zrenner E, Messias A et al: Transcorneal Electrical Stimulation for Patients With Retinitis Pigmentosa: A Prospective, Randomized, Sham-Controlled Follow-up Study Over 1 Year. Invest Ophthalmol Vis Sci 2017, 58(1):257-269.
57. Sehic A, Guo S, Cho K S, Corraya R M, Chen D F, Utheim T P. Electrical Stimulation as a Means for Improving Vision. Am J Pathol 2016; 186:2783-2797.
58. Seki M, Tanaka T, Sakai Y, et al. Muller Cells as a source of brain-derived neurotrophic factor in the retina: noradrenaline upregulates brain-derived neurotrophic factor levels in cultured rat Muller cells. Neurochem Res 2005; 30:1163-1170.
59. Shi C, Yuan X, Chang K, et al. Optimization of Optomotor Response-based Visual Function Assessment in Mice. Sci Rep 2018; 8:9708.
60. Shi C, Yuan X, Chang K, et al. Optimization of Optomotor Response-based Visual Function Assessment in Mice. Scientific Reports 2018; 8:9708.
61. Shimozawa A, Furuta E: Ganglion cells in the facial nerve trunk of the mouse. Anat Anz 1979, 146(2):133-140.
62. Shinoda K, Imamura Y, Matsuda S, Seki M, Uchida A, Grossman T, Tsubota K: Transcutaneous electrical retinal stimulation therapy for age-related macular degeneration. Open Ophthalmol J 2008, 2:132-136.
63. Spurr-Michaud S, Argueso P, Gipson I: Assay of mucins in human tear fluid. Exp Eye Res 2007, 84(5):939-950.
64. Wang X, Mo X, Li D, et al. Neuroprotective effect of transcorneal electrical stimulation on ischemic damage in the rat retina. Exp Eye Res 2011; 93:753-760.
65. Willcox M D P, Argueso P, Georgiev G A, Holopainen J M, Laurie G W, Millar T J, Papas E B, Rolland J P, Schmidt T A, Stahl U et al: TFOS DEWS I I Tear Film Report. Ocul Surf 2017, 15(3):366-403.
66. Yan T, Jiang X, Guo X, Chen W, Tang D, Zhang J, Zhang X, Zhang D, Zhang Q, Jia J et al: Electric field-induced suppression of PTEN drives epithelial-to-mesenchymal transition via mTORC 1 activation. J Dermatol Sci 2017, 85(2):96-105.
67. Yang M, Botten N, Hodges R, Bair J, Utheim T P, Serhan C N, Dartt D A: Resolvin D2 and Resolvin D1 Differentially Activate Protein Kinases to Counter-Regulate Histamine-Induced [Ca(2+)+]ᵢ Increase and Mucin Secretion in Conjunctival Goblet Cells. International journal of molecular sciences 2021, 23(1).
68. Yang M, Lippestad M, Hodges R R, Fjaervoll H K, Fjaervoll K A, Bair J A, Utheim T P, Serhan C N, Dartt D A: RvE1 uses the LTB4 receptor BLT1 to increase [Ca(2+)+]ᵢ and stimulate mucin secretion in cultured rat and human conjunctival goblet cells. Ocul Surf 2020, 18(3):470-482.
69. Yin H, Yin H, Zhang W, et al. Transcorneal electrical stimulation promotes survival of retinal ganglion cells after optic nerve transection in rats accompanied by reduced microglial activation and TNF-alpha expression. Brain Res 2016:1650:10-20.
70. You J S, Anderson G B, Dooley M S, Homberger T A: The role of mTOR signaling in the regulation of protein synthesis and muscle mass during immobilization in mice. Dis Model Mech 2015, 8(9):1059-1069.
71. Yu H, Enayati S, Chang K, Cho K, Lee S W, Talib M, Zihlavnikova K, Xie J, Achour H, Fried S I et al: Noninvasive Electrical Stimulation Improves Photoreceptor Survival and Retinal Function in Mice with Inherited Photoreceptor Degeneration. Invest Ophthalmol Vis Sci 2020, 61(4):5.
72. Yu H, Enayati S, Chang K, et al. Noninvasive Electrical Stimulation Improves Photoreceptor Survival and Retinal Function in Mice with Inherited Photoreceptor Degeneration. Invest Ophthalmol Vis Sci 2020; 61:5.
73. Yu W S, Kwon S H, Agadagba S K, Chan L L, Wong K H, Lim L W: Neuroprotective Effects and Therapeutic Potential of Transcorneal Electrical Stimulation for Depression. Cells 2021, 10(9).
74. Zhao L, Ma W, Fariss R N, Wong W T. Minocycline attenuates photoreceptor degeneration in a mouse model of subretinal hemorrhage microglial: inhibition as a potential therapeutic strategy. Am J Pathol 2011; 179:1265-1277.
75. Zheng Y, Jiang Z, Ping A, Zhang F. Zhu J, Wang Y, Zhu W, Xu K: Acute Seizure Control Efficacy of Multi-Site Closed-Loop Stimulation in a Temporal Lobe Seizure Model. IEEE Trans Neural Syst Rehabil Eng 2019, 27(3):419-428.
76. Zhou W T, Ni Y Q, Jin Z B, et al. Electrical stimulation ameliorates light-induced photoreceptor degeneration in vitro via suppressing the proinflammatory effect of microglia and enhancing the neurotrophic potential of Muller cells. Exp Neurol 2012; 238:192-208.

1. A method of treating macular degeneration, age-related macular degeneration, photoreceptor degeneration, ischemic retinopathy, diabetic retinopathy, ischemic optic neuropathy, traumatic optic neuropathy, and/or glaucoma in a subject comprising delivering electrical stimulation in a ramp waveform to the eye of the subject.

2. A method of treating corneal abrasions, corneal ulcers, dry eye syndrome, corneal neuropathic pain, neuropathic keratitis, and/or corneal vascularization in a subject comprising delivering electrical stimulation in a ramp waveform to an eye of the subject.

3. (canceled)

4. A method of preserving visual function in an injured or diseased eye of a subject comprising delivering electrical stimulation in a ramp waveform to the eye of the subject.

5. (canceled)

6. (canceled)

7. (canceled)

8. The method of claim 1, wherein the electrical stimulation is delivered transpalpebrally.

9. The method of claim 1, wherein the ramp waveform is approximately 100 ms in duration.

10. The method of claim 1, wherein the ramp waveform is delivered at approximately 20 Hz.

11. The method of claim 1, wherein the electrical stimulation comprises both a ramp waveform and a rectangular waveform.

12. The method of claim 1, wherein the electrical stimulation is delivered every day or every other day.

13. The method of claim 1, wherein the electrical stimulation is delivered at least three days at least four days, at least five days, or at least six days per week.

14. The method of claim 1, wherein the electrical stimulation further comprises days in which no electrical stimulation is delivered.

15. The method of claim 1, wherein the electrical stimulation is delivered for at least one minute, two minutes, three minutes, four minutes, five minutes, six minutes, seven minutes, eight minutes, nine minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes, or 30 minutes per day.

16. The method of claim 1, wherein the electrical stimulation is delivered for no more than 30 minutes, 29 minutes, 28 minutes, 27 minutes, 26 minutes, 25 minutes, 24 minutes, 23 minutes, 22 minutes, 21 minutes, 20 minutes, 19 minutes, 18 minutes, 17 minutes 16 minutes, 15 minutes, 14 minutes, 13 minutes, 12 minutes, 11 minutes, 10 minutes, nine minutes, eight minutes, seven minutes, six minutes, five minutes, four minutes, three minutes, two minutes, or one minute per day.

17. The method of claim 1, wherein the electrical stimulation comprises amplitude priority stimulation.

18. The method of claim 1, wherein the electrical stimulation comprises about 100 μA to about 500 μA.

19. The method of claim 1, wherein the electrical stimulation comprises about 300 μA.

20. The methods of claim 1, wherein the electrical stimulation comprises about 100 μA.

21. The method of claim 4, wherein the preserving visual function in a subject comprises preserving visual acuity and/or contrast sensitivity.

22. The method of claim 4, wherein the preserving visual function in a subject comprises preserving positive scotopic threshold response in the subject.

23. The method of claim 4, wherein the preserving visual function in a subject comprises preserving an ERG a wave in a subject.

24. The method of claim 6, wherein the repressing microglia activation comprises repressing angiogenic processes and/or inducing anti-angiogenic processes.
